(19) United States
(12) Patent Application Publication
(10) Pub. No.: US 2024/0325069 A1
(43) Pub. Date: Oct. 3, 2024

(54) Electrosurgical Devices Having Embedded Sensors, Methods of Use, and Methods of Manufacture

(71) Applicant: Stryker European Operations Limited, Cork (IE)

(72) Inventors: Laura Constance Frey, Belfast (IE); Scott McFarland, Greenisland (IE); Paul Sheridan, Wexford (IE); Micheal Burke, Cork (IE)

(21) Appl. No.: 18/693,824

(22) PCT Filed: Oct. 5, 2022
(86) PCT No.: PCT/IB2022/000575

§ 371 Date: Mar. 20, 2024

Related U.S. Application Data
(60) Provisional application No. 63/252,549, filed on Oct. 5, 2021.

(51) Int. Cl. A61B 18/12; A61B 17/00; A61B 18/00; A61B 18/14; G03F 7/00

(52) U.S. Cl. CPC A61B 18/1206; A61B 18/148; G03F 7/0017; A61B 2017/00066; A61B 2017/00526; A61B 2018/00083; A61B 2018/00178; A61B 2018/00601; A61B 2018/00702; A61B 2018/00821; A61B 2018/00875; A61B 2018/00898; A61B 2018/00904; A61B 2018/1253; A61B 2018/1412

(57) ABSTRACT

In an example, a monopolar electrosurgical electrode includes an electrosurgical substrate including an electrically conductive material extending in an axial direction from a proximal end to a distal end. The electrosurgical substrate includes an electrosurgical blade. The electrosurgical blade includes (i) a first lateral surface, (ii) a second lateral surface opposite the first lateral surface, (iii) a first major surface extending between the first lateral surface and the second lateral surface on a first side of the electrosurgical blade, and (iv) a second major surface extending between the first lateral surface and the second lateral surface on a second side of the electrosurgical blade that is opposite the first side. The monopolar electrosurgical electrode also includes a first electrode sensor embedded between a plurality of electrical insulation layers on the first major surface of the electrosurgical blade.

### CROSS-REFERENCE TO RELATED APPLICATIONS

This application claims the benefit of U.S. Provisional Application No. 63/252,549, filed Oct. 5, 2021, the contents of which are hereby incorporated by reference in their entirety.

### FIELD

The present disclosure generally relates to electrosurgical devices and, more specifically, to electrosurgical devices and the methods for sensing an operational conditions during an electrosurgical procedure.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

### SUMMARY

In an example, a monopolar electrosurgical electrode includes an electrosurgical substrate including an electrically conductive material extending in an axial direction from a proximal end to a distal end. The electrosurgical substrate includes an electrosurgical blade. The electrosurgical blade includes (i) a first lateral surface, (ii) a second lateral surface opposite the first lateral surface, (iii) a first major surface extending between the first lateral surface and the second lateral surface on a first side of the electrosurgical blade, and (iv) a second major surface extending between the first lateral surface and the second lateral surface on a second side of the electrosurgical blade that is opposite the first side. The monopolar electrosurgical electrode also includes a first electrode sensor embedded between a plurality of electrical insulation layers on the first major surface of the electrosurgical blade.

In another example, a method of forming a monopolar electrosurgical electrode is described. The method includes forming an electrosurgical substrate from an electrically conductive material. The electrosurgical substrate includes an electrosurgical blade. The electrosurgical blade includes a first lateral surface, a second lateral surface opposite the first lateral surface, a first major surface extending between the first lateral surface and the second lateral surface on a first side of the electrosurgical blade, and a second major surface extending between the first lateral surface and the second lateral surface on a second side of the electrosurgical blade that is opposite the first side.

The method can also include forming a first electrical insulation layer on the first major surface the electrosurgical blade. The method can further include forming a first electrode sensor on the first electrical insulation layer on the first major surface. The method can include forming a second electrical insulation layer on the first electrode sensor and the first electrical insulation layer such that the first electrode sensor is embedded between the first electrical insulation layer and the second electrical insulation layer.

In another example, a method for performing electrosurgery is described. The method includes coupling an electrosurgical tool to an electrosurgical generator. The electrosurgical tool includes a monopolar electrosurgical electrode. The monopolar electrosurgical electrode includes an electrosurgical substrate including an electrically conductive material extending in an axial direction from a proximal end to a distal end. The proximal end is configured to receive electrosurgical energy from an electrosurgical tool. The electrosurgical substrate includes an electrosurgical blade that is configured for at least one of cutting or coagulation of tissue by the electrosurgical energy received from the electrosurgical tool.

The electrosurgical blade includes: (i) a first lateral surface, (ii) a second lateral surface opposite the first lateral surface, (iii) a first major surface extending between the first lateral surface and the second lateral surface on a first side of the electrosurgical blade, (iv) a second major surface extending between the first lateral surface and the second lateral surface on a second side of the electrosurgical blade that is opposite the first side, and (v) a first electrode sensor embedded between a plurality of electrical insulation layers on the first major surface of the electrosurgical blade.

The method also includes receiving, by the electrosurgical tool, the electrosurgical energy from the electrosurgical generator. Responsive to receiving the electrosurgical energy, the method includes performing, using the monopolar electrosurgical electrode, an electrosurgical operation. The method further includes sensing, using the first electrode sensor, a condition related to the electrosurgical operation.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 depicts a simplified block diagram of an electrosurgical system, according to an example.

FIG. 2 depicts a cross-sectional view of an electrosurgical tool, according to an example.

FIG. 3A depicts a first side view of an electrosurgical tool, according to an example.

FIG. 3B depicts a second side view of the electrosurgical tool shown in FIG. 3A, according to an example.

FIG. 3C depicts a cross-sectional view of the electrosurgical tool shown in FIG. 3B, according to an example.

FIG. 3D depicts a cross-sectional view of a monopolar electrosurgical electrode shown in FIG. 3A, according to an example.

FIG. 3E depicts a first perspective view of the monopolar electrosurgical electrode shown in FIG. 3A, according to an example.

FIG. 3F depicts a second perspective view of the monopolar electrosurgical electrode shown in FIG. 3A, according to an example.

FIG. 3G depicts a receptacle including one or more receptacle contacts for coupling with one or more electrode contacts on a shank portion of the monopolar electrosurgical electrode, according to an example.

FIG. 3H depicts the sensor contacts of the shank portion of the monopolar electrosurgical electrode electrically coupled to the receptacle contacts of the receptacle, according to an example.

FIG. 4 depicts the monopolar electrosurgical electrode, according to another example.

FIG. 5 depicts a perspective view of an electrosurgical tool, according to another example.

FIG. 6A depicts a first view of a wafer for batch fabrication of a plurality of monopolar electrosurgical electrodes, according to an example.

FIG. 6B depicts a second view of the wafer shown in FIG. 6A, according to an example.

FIG. 7A depicts a cross-sectional view of an electrosurgical blade at a first stage of a fabrication process, according to an example.

FIG. 7B depicts a cross-sectional view of an electrosurgical blade at a first stage of a fabrication process, according to an example.

FIG. 7C depicts a cross-sectional view of an electrosurgical blade at a first stage of a fabrication process, according to an example.

FIG. 7D depicts a cross-sectional view of an electrosurgical blade at a first stage of a fabrication process, according to an example.

FIG. 7E depicts a cross-sectional view of an electrosurgical blade at a first stage of a fabrication process, according to an example.

FIG. 7F depicts a cross-sectional view of an electrosurgical blade at a first stage of a fabrication process, according to an example.

FIG. 8 depicts a flow chart of a process for forming a monopolar electrosurgical electrode according to an example.

FIG. 9 depicts a flow chart of a process for forming a monopolar electrosurgical electrode that can be used with at least the process shown in FIG. 8, according to an example.

FIG. 10 depicts a flow chart of a process for forming a monopolar electrosurgical electrode that can be used with at least the process shown in FIG. 8, according to an example.

FIG. 11 depicts a flow chart of a process for forming a monopolar electrosurgical electrode that can be used with at least the process shown in FIG. 8, according to an example.

FIG. 12 depicts a flow chart of a process for forming a monopolar electrosurgical electrode that can be used with at least the process shown in FIG. 11, according to an example.

FIG. 13 depicts a flow chart of a process for forming a monopolar electrosurgical electrode that can be used with at least the process shown in FIG. 12, according to an example.

FIG. 14 depicts a flow chart of a process for forming a monopolar electrosurgical electrode that can be used with at least the process shown in FIG. 8, according to an example.

FIG. 15 depicts a flow chart of a process for forming a monopolar electrosurgical electrode that can be used with at least the process shown in FIG. 8, according to an example.

FIG. 16 depicts a flow chart of a process for forming a monopolar electrosurgical electrode that can be used with at least the process shown in FIG. 15, according to an example.

FIG. 17 depicts a flow chart of a process for forming a monopolar electrosurgical electrode that can be used with at least the process shown in FIG. 8, according to an example.

FIG. 18 depicts a flow chart of a process for performing electrosurgery according to an example.

FIG. 19 depicts a flow chart of a process for performing electrosurgery that can be used with at least the process shown in FIG. 18, according to an example.

FIG. 20 depicts a flow chart of a process for performing electrosurgery that can be used with at least the process shown in FIG. 19, according to an example.

FIG. 21 depicts a flow chart of a process for performing electrosurgery that can be used with at least the process shown in FIG. 20, according to an example.

FIG. 22 depicts a flow chart of a process for performing electrosurgery that can be used with at least the process shown in FIG. 18, according to an example.

FIG. 23 depicts a flow chart of a process for performing electrosurgery that can be used with at least the process shown in FIG. 18, according to an example.

FIG. 24 depicts a flow chart of a process for performing electrosurgery that can be used with at least the process shown in FIG. 18, according to an example.

FIG. 25 depicts a flow chart of a process for performing electrosurgery that can be used with at least the process shown in FIG. 18, according to an example.

FIG. 26 depicts a flow chart of a process for performing electrosurgery that can be used with at least the process shown in FIG. 18, according to an example.

FIG. 27 depicts a flow chart of a process for performing electrosurgery that can be used with at least the process shown in FIG. 18, according to an example.

FIG. 28 depicts a flow chart of a process for performing electrosurgery that can be used with at least the process shown in FIG. 18, according to an example.

FIG. 29 depicts a flow chart of a process for performing electrosurgery that can be used with at least the process shown in FIG. 18, according to an example.

FIG. 30 depicts a flow chart of a process for performing electrosurgery that can be used with at least the process shown in FIG. 18, according to an example.

FIG. 31 depicts a flow chart of a process for performing electrosurgery that can be used with at least the process shown in FIG. 30, according to an example.

FIG. 32 depicts a flow chart of a process for performing electrosurgery that can be used with at least the process shown in FIG. 18, according to an example.

FIG. 33 depicts a flow chart of a process for performing electrosurgery that can be used with at least the process shown in FIG. 32, according to an example.

FIG. 34 depicts a flow chart of a process for performing electrosurgery that can be used with at least the process shown in FIG. 18, according to an example.

FIG. 35 depicts a flow chart of a process for performing electrosurgery that can be used with at least the process shown in FIG. 18, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, an electrosurgical device can use electrical energy supplied by an electrosurgical generator to apply electrosurgical energy from an electrosurgical electrode to a tissue. Among other things, it can be beneficial for the electrosurgical device to selectively remove a fixed quantity of tissue at a specific location while reducing or minimizing blood loss, smoke generation, dead tissue build up, thermal spreading, and/or scarring. Existing monopolar electrosurgical devices generally have limited information relating to operating conditions associated with a target tissue and/or an electrosurgical electrode during the electrosurgical procedure. As a result, existing monopolar electrosurgical devices during electrosurgical procedures may be limited with respect to one or more of the performance metrics noted above.

The present application provides for monopolar electrosurgical electrodes that can be incorporated into an electrosurgical tool to address one or more of the challenges described above. In particular, the present application provides for a monopolar electrosurgical electrode in the form of an electrosurgical blade that includes one or more sensors embedded in the electrosurgical blade. The sensor(s) can sense one or more operational conditions during an electrosurgical procedure, and transmit sensor signals that can provide a basis for feedback control of an electrosurgical system to improve the electrosurgical procedure.

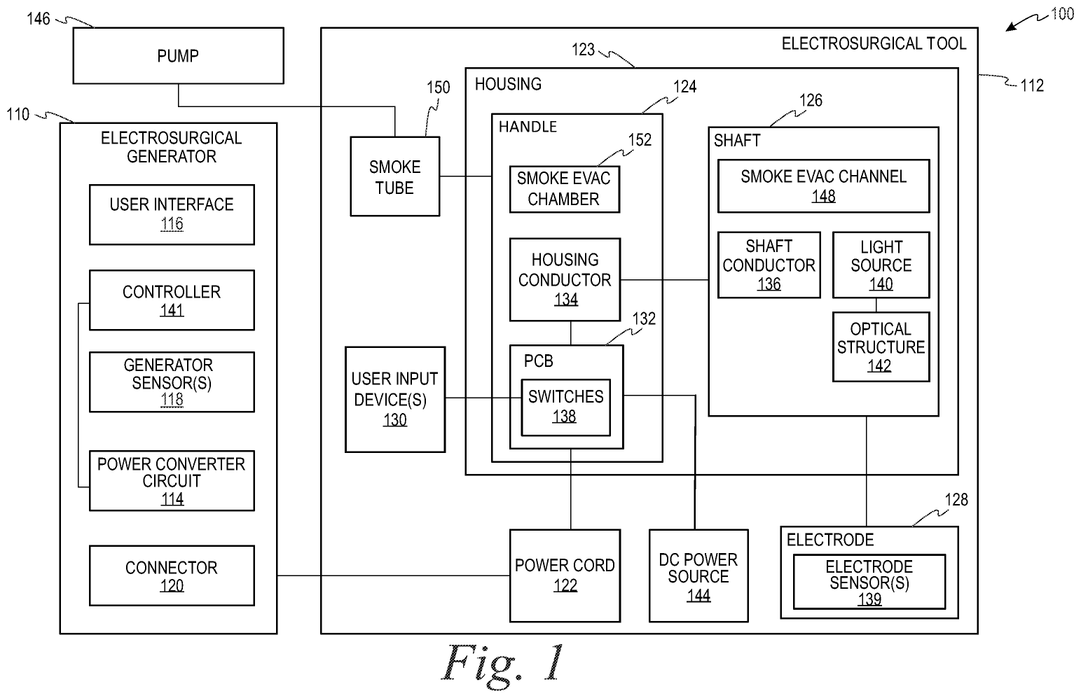

Referring now to FIG. 1, an electrosurgical system 100 is shown according to an example. As shown in FIG. 1, the electrosurgical system 100 includes an electrosurgical generator 110 and an electrosurgical tool 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, one or more display screens, one or more indicator lights, one or more speakers, and/or one or more haptic output devices.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more generator sensors 118 that can sense one or more conditions related to the electrosurgical energy and/or the target tissue. As examples, the generator sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the generator sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 kHz.

In FIG. 1, the electrosurgical generator 110 also includes a connector 120 that can facilitate coupling the electrosurgical generator 110 to the electrosurgical tool 112. For example, the electrosurgical tool 112 can include a power cord 122 having a plug, which can be coupled to a socket of the connector 120 of the electrosurgical generator 110. In this arrangement, the electrosurgical generator 110 can supply the electrosurgical energy to the electrosurgical tool 112 via the coupling between the connector 120 of the electrosurgical generator 110 and the power cord 122 of the electrosurgical tool 112.

The electrosurgical generator 110 can further include a controller 141 that can control operation of the electrosurgical generator 110. Within examples, the controller 141 can be implemented using hardware, software, and/or firmware. For instance, the controller 141 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 141, thus, can receive data and store the data in the memory as well. As shown in FIG. 1, the controller 141 can be communicatively coupled with the power converter circuit 114, the user interface 116, the generator sensor(s) 118, and/or the connector 120.

As shown in FIG. 1, the electrosurgical tool 112 can include a housing 123. The housing 123 can be an elongated structure in and/or on which components of the electrosurgical tool 112 can be disposed. In some examples, the housing 123 can be an integral, monolithic structure. In other examples the housing 123 can include a plurality of structures that are coupled to each other.

In FIG. 1, the housing 123 includes a handle 124 that defines an interior bore, a shaft 126 extending in a distal direction from the handle 124, and a monopolar electrosurgical electrode 128 extending in the distal direction from the shaft 126. In general, the handle 124 can be configured to facilitate a user gripping and manipulating the electrosurgical tool 112 while performing electrosurgery. For example, the handle 124 can have a shape and/or a size that can facilitate a user performing electrosurgery by manipulating the electrosurgical tool 112 using a single hand. In one implementation, the handle 124 can have a shape and/or a size that facilitates the user holding the electrosurgical tool 112 in a writing utensil gripping manner (e.g., the electrosurgical tool 112 can be an electrosurgical pencil).

Additionally, for example, the handle 124 and/or the shaft 126 can be constructed from one or more materials that are electrical insulators (e.g., a plastic material). This can facilitate insulating the user from the electrosurgical energy flowing through the electrosurgical tool 112 while performing the electrosurgery.

In some implementations, the shaft 126 can be coupled to the handle 124 in a fixed and non-moveable manner. This may simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). In one example, the handle 124 and the shaft 126 can be formed as a single, monolithic structure such that the shaft 126 and the handle 124 are fixed and non-moveable relative to each other. In another example, the handle 124 and the shaft 126 can be fixedly coupled to each other by a welding coupling, an adhesive coupling, and/or another coupling that prevents movement between the handle 124 and the shaft 126.

In other implementations, the shaft 126 can be telescopically moveable relative to the handle 124. For example, the shaft 126 can be telescopically moveable in the interior bore defined by the handle 124 to extend the shaft 126 in the distal direction and retract the shaft 126 in a proximal direction relative to the handle 124 (e.g., movable along a longitudinal axis of the electrosurgical tool 112). In some examples, the monopolar electrosurgical electrode 128 can be coupled to the shaft 126 and, thus, the monopolar electrosurgical electrode 128 can move together with the shaft 126 in an axial direction along the longitudinal axis relative to the handle 124. This can provide for adjusting a length of the electrosurgical tool 112, which can facilitate performing electrosurgery at a plurality of different depths within tissue (e.g., due to different anatomical shapes and/or sizes of patients) and/or at a plurality of different angles.

In some implementations, the monopolar electrosurgical electrode 128 can additionally or alternatively be rotatable about an axis of rotation that is parallel to the longitudinal axis of the electrosurgical tool 112. In some examples, the monopolar electrosurgical electrode 128 can be rotatable relative to the handle 124 and the shaft 126. In other examples, the monopolar electrosurgical electrode 128 can be rotationally fixed relative to the shaft 126 such that the shaft 126 and the monopolar electrosurgical electrode 128 are rotatable together relative to the handle 124. Rotating the monopolar electrosurgical electrode 128 relative to the handle 124 can facilitate adjusting an angle of the monopolar electrosurgical electrode 128 relative to one or more user input device(s) 130 of the electrosurgical tool 112. In this arrangement, a user can comfortably grip the handle 124 in a position in which their fingers can comfortably operate the user input device(s) 130 while the monopolar electrosurgical electrode 128 is set at a rotational position selected from among a plurality of rotational positions relative to the handle 124 based on, for example, a location, a size, and/or a shape of a surgical site in which the user is operating.

In one implementation, the monopolar electrosurgical electrode 128 can be rotatable by more than 360 degrees relative to the handle 124. This can improve an ease of use by allowing an operator to freely rotate the monopolar electrosurgical electrode 128 without limitation. However, in other implementations, the monopolar electrosurgical electrode 128 can be rotatable by less than or equal to 360 degrees (e.g., rotatable by 180 degrees or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

Although it can be beneficial to provide for rotation of the monopolar electrosurgical electrode 128 relative to the handle 124 and/or the shaft 126, the monopolar electrosurgical electrode 128 can be rotationally fixed relative to the handle 124 and the shaft 126 in some implementations. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts).

The user input device(s) 130 can select between the modes of operation of the electrosurgical tool 112 and/or the electrosurgical generator 110. For instance, in one implementation, the user input device(s) 130 can be configured to select between a cutting mode of operation and a coagulation mode of operation. Responsive to actuation of the user input device(s) 130 of the electrosurgical tool 112, the electrosurgical tool 112 can (i) receive the electrosurgical energy with a level of power and/or a waveform corresponding to the mode of operation selected via the user input device(s) 130 and (ii) supply the electrosurgical energy to the monopolar electrosurgical electrode 128.

In FIG. 1, the electrosurgical tool 112 includes a plurality of electrical components that facilitate supplying the electrosurgical energy, which the electrosurgical tool 112 receives from the electrosurgical generator 110, to the monopolar electrosurgical electrode 128. For example, the electrosurgical tool 112 can include at least one electrical component selected from a group of electrical components including: a printed circuit board 132 (e.g., a flexible printed circuit board), a housing conductor 134, and/or a shaft conductor 136 that can provide a circuit for conducting the electrosurgical energy from the power cord 122 to the monopolar electrosurgical electrode 128. One or more of the electrical components can be positioned in the inner bore defined by the handle 124 and/or in the inner cavity defined by the shaft 126.

Within examples, the user input device(s) 130 can include one or more buttons on an exterior surface of the handle 124. Each button of the user input device(s) 130 can be operable to actuate a respective one of a plurality of switches 138 of the printed circuit board 132. In general, the switches 138 and/or the printed circuit board 132 are operable to control a supply of the electrosurgical energy from the electrosurgical generator 110 to the monopolar electrosurgical electrode 128. For instance, in one implementation, when each button is operated (e.g., depressed), the respective switch 138 associated with the button can be actuated to cause the printed circuit board 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 138 associated with the button can close the switch 138 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the printed circuit board 132 can be omitted.

In both example implementations, the electrosurgical energy supplied by the electrosurgical generator 110 can be supplied from (i) the power cord 122, the printed circuit board 132, and/or the switches 138 to (ii) the monopolar electrosurgical electrode 128 by the housing conductor 134 and the shaft conductor 136. As such, as shown in FIG. 1, the printed circuit board 132 can be coupled to the power cord 122, the housing conductor 134 can be coupled to the printed circuit board 132 and the shaft conductor 136, and the shaft conductor 136 can be coupled to the monopolar electrosurgical electrode 128. In this arrangement, the housing conductor 134 can conduct the electrosurgical energy (supplied to the housing conductor 134 via the printed circuit board 132) to the shaft conductor 136, and the shaft conductor 136 can conduct the electrosurgical energy to the monopolar electrosurgical electrode 128.

In general, the housing conductor 134 and the shaft conductor 136 can each include one or more electrically conductive elements that provide an electrically conductive bus for supplying the electrosurgical energy to the monopolar electrosurgical electrode 128. More particularly, the housing conductor 134 can include one or more electrically conductive elements of the handle 124 that can supply the electrosurgical energy to the shaft conductor 136, and the shaft conductor 136 can include one or more electrically conductive elements of the shaft 126 that can supply the electrical energy from the housing conductor 134 to the monopolar electrosurgical electrode 128. In implementations in which the shaft 126 is movable or rotatable relative to the handle 124, the housing conductor 134 can engage the shaft conductor 136 to maintain an electrical coupling between the housing conductor 134, the shaft conductor 136, and the monopolar electrosurgical electrode 128 while (i) the shaft 126 and/or the monopolar electrosurgical electrode 128 telescopically moves relative to the handle 124, and/or (ii) the monopolar electrosurgical electrode 128 rotates relative to the handle 124.

Although the electrosurgical tool 112 includes the user input device(s) 130 in FIG. 1, the user input device(s) 130 can be separate from the electrosurgical tool 112 in another example. For instance, the user input device(s) 130 can additionally or alternatively include one or more foot pedals that are actuatable to control operation of the electrosurgical tool 112 as described above. The foot pedal(s) can be communicatively coupled to the electrosurgical generator 110 to provide a signal responsive to actuation of the foot pedal(s).

In some examples, the electrosurgical tool 112 can additionally include one or more light sources 140 that are configured to emit light. In some implementations, the light source(s) 140 can be located at a distal end of the housing 123 and/or a distal end of the shaft 126 to directly provide light in a distal direction and illuminate a surgical distal of the monopolar electrosurgical electrode 128.

In other implementations, as shown in FIG. 1, the light source(s) 140 can be optically coupled to an optical structure 142, which is configured to receive the light emitted by the light source(s) 140 and transmit the light in a distal direction toward a surgical site to illuminate the surgical site while performing electrosurgery using the monopolar electrosurgical electrode 128. Although arranging the light source(s) 140 to directly illuminate a surgical field can help, for instance, to reduce a cost of manufacture, transmitting the light using the optical structure 142 can help to improve a quality of light transmitted from the electrosurgical tool 112 (e.g., by providing light with improved uniformity and/or reduced heat generation).

As examples, in implementations that include the optical structure 142, the optical structure 142 can include at least one optical structure selected from among a group consisting of an optical lens, a non-fiber optic optical waveguide, and an optical fiber. When the optical structure 142 includes the optical lens (e.g., a parabolic reflector lens, an aspheric lens, and/or a Fresnel lens), the optical structure 142 can help to direct the light emitted by the light source 140 in the distal direction and thereby improve a quality of the light illuminating the surgical site. The optical structure 142 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 142 can additionally or alternatively include other light shaping optical elements such as, for instance, a plurality of facets, one or more prisms, and/or one or more optical gratings. Although the optical structure 142 can help to improve a quality of the light directed to the surgical site, the electrosurgical tool 112 can omit the optical structure 142 and instead emit the light from the light source 140 directly to the surgical field without transmitting the light through the optical structure 142 in other examples.

In FIG. 1, the light source 140 can be coupled to the shaft 126. As such, the light source 140 can also move telescopically with the shaft 126 relative to the handle 124. However, in other examples, the light source 140 can be in the interior bore of the handle 124 and/or coupled to an exterior surface of the handle 124. As examples, the light source 140 can include one or more light emitting diodes (LEDs), organic light emitting diodes (OLEDs), optical fibers, non-fiber optic waveguides, and/or lenses. Additionally, for example, the light source 140 can include a light-emitting diode printed circuit board (LED PCB) having one or more light sources (e.g., LEDs). As described in further detail below, the LED PCB can include a PCB aperture, and one or more other components (e.g., the monopolar electrosurgical electrode 128) of the electrosurgical tool 112 can extend through the aperture.

The optical structure 142 can be at a distal end of the shaft 126. In some examples, the optical structure 142 can circumferentially surround the monopolar electrosurgical electrode 128 to emit the light distally around all sides of the monopolar electrosurgical electrode 128. This can help to mitigate shadows and provide greater uniformity of illumination in all rotational alignments of the shaft 126 relative to the housing 123 and/or the electrosurgical tool 112 relative to the target tissue. However, in other examples, the optical structure 142 can extend partially but not fully around the monopolar electrosurgical electrode 128.

In implementations that include the light source 140, the user input device(s) 130, the printed circuit board 132, the switches 138, the housing conductor 134, and/or the shaft conductor 136 can additionally supply an electrical power from a direct current (DC) power source 144 to the light source 140. In one example, the DC power source 144 can include a battery disposed in the handle 124, the plug of the power cord 122, and/or a battery receptacle located along the power cord 122 between the handle 124 and the plug. Although the electrosurgical tool 112 includes the DC power source 144 in FIG. 1, the DC power source 144 can be separate and distinct from the electrosurgical tool 112 in other examples. For instance, in another example, the electrosurgical generator 110 can include the DC power source 144.

Additionally, in implementations that include the light source 140, the user input device(s) 130 can be operable to cause the light source 140 to emit the light. In one example, the user input device(s) 130 can include a button that independently controls the light source 140 separate from the button(s) that control the electrosurgical operational modes of the electrosurgical tool 112. In another example, the user input device(s) 130 and the printed circuit board 132 can be configured such that operation of the button(s) that control the electrosurgical operational mode simultaneously control operation of the light source 140 (e.g., the light source 140 can be automatically actuated to emit light when a button is operated to apply the electrosurgical energy at the monopolar electrosurgical electrode 128).

As shown in FIG. 1, responsive to operation of the user input device(s) 130 to actuate the light source 140, the DC power source 144 can supply the electrical power (e.g., a DC voltage) to the light source 140 via the printed circuit board 132, the housing conductor 134, and/or the shaft conductor 136. In this implementation, one or more of the conductive elements of the housing conductor 134 can be configured to supply the electrical power from the DC power source 144 to the light source 140 and/or return the electrical power from the light source 140 to the DC power source 144. Accordingly, the housing conductor 134 can additionally or alternatively assist in providing electrical communication between the DC power source 144 and the light source 140 as the shaft 126 and the light source 140 telescopically move relative to the handle 124.

Although the user input device(s) 130 on the handle 124 can be operated to control the operation of the light source 140 in the examples described above, the light source 140 can be additionally or alternatively operated by one or more user input device(s) on the electrosurgical generator 110 (e.g., via the user interface 116) and/or on the plug of the power cord 122.

Within examples, the electrosurgical tool 112 can additionally or alternatively include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump 146 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 146 and the electrosurgical generator 110 can be provided as separate devices or integrated in a single device (e.g., in a common housing).

As shown in FIG. 1, the shaft 126 can include a smoke evacuation channel 148 in the inner cavity of the shaft 126. The smoke evacuation channel 148 can also include a smoke inlet that can extend circumferentially around a center axis of a distal portion of the monopolar electrosurgical electrode 128. In this arrangement, the smoke inlet of the smoke evacuation channel can help to receive surgical smoke into the smoke evacuation channel 148 in all rotational alignments of the monopolar electrosurgical electrode 128 relative to the handle 124 and/or the electrosurgical tool 112 relative to the target tissue. However, in another example, the smoke evacuation channel 148 can include one or more smoke inlets that do not extend circumferentially around the monopolar electrosurgical electrode 128.

In an example, the smoke evacuation channel 148 can include an outer tube that is separated from the optical structure 142 by an air gap. For instance, the shaft 126 can include a plurality of standoffs that extend between the optical structure 142 and the outer tube of the smoke evacuation channel 148 to provide the air gap between the outer tube and the optical structure 142. In one implementation, the optical structure 142 can include the standoffs such that the optical structure 142 and the standoffs are formed as a single, monolithic structure. In another implementation, the standoffs can be formed as a single, monolithic structure with the outer tube of the smoke evacuation channel 148. In another implementation, the standoffs can be separate from the outer tube of the smoke evacuation channel 148 and the optical structure 142.

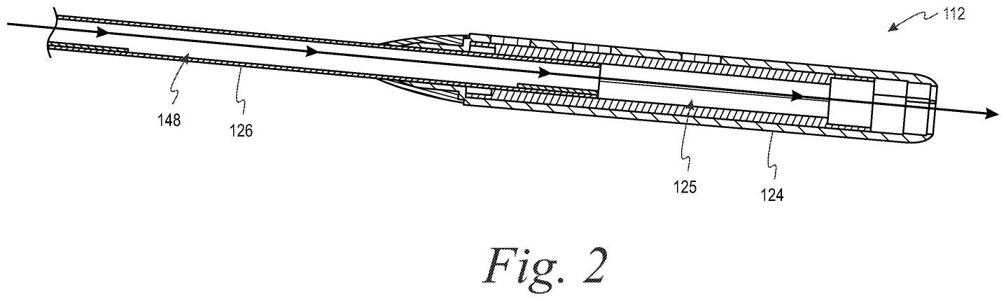

In an example, the smoke evacuation channel 148 of the shaft 126 defines a first portion of a smoke flow path, and the interior bore 125 of the handle 124 defines a second portion of a smoke flow path. FIG. 2 illustrates a partial cross-sectional view of the electrosurgical tool 112 according to an implementation of this example. In this arrangement, the surgical smoke can be received from the surgical site into the smoke evacuation channel 148 of the shaft 126, and flow proximally along the smoke evacuation channel 148 to the interior bore 125 of the handle 124. In the interior bore 125 of the handle 124, the smoke can further flow to a smoke tube 150 that is coupled to a proximal end of the handle 124 and configured to convey smoke from the handle 124 to the suction pump 146.

As noted above, the monopolar electrosurgical electrode 128 can apply the electrosurgical energy to a target tissue to perform an electrosurgical operation (e.g., cutting, coagulating, ablating, and/or sealing the target tissue). Within examples, the monopolar electrosurgical electrode 128 includes an electrosurgical substrate formed from an electrically conductive material. As an example, the electrically conductive material can be stainless steel.

As described in further detail below, the electrosurgical substrate can extend in an axial direction from a proximal end of the monopolar electrosurgical electrode 128 to a distal end of the monopolar electrosurgical electrode 128. The proximal end of the monopolar electrosurgical electrode 128 can receive electrosurgical energy from the electrosurgical tool 112 (e.g., via the housing conductor 134 and the shaft conductor 136 as described above), and a distal working portion of the monopolar electrosurgical electrode 128 can apply the electrosurgical energy to the target tissue. In one implementation, the electrosurgical substrate can include a shank portion that extends from the proximal end of monopolar electrosurgical electrode 128 to the distal working portion of the monopolar electrosurgical electrode 128. The distal working portion can be configured for at least one of cutting or coagulation of tissue by the electrosurgical energy received from the electrosurgical tool 112.

In some examples, the distal working portion can define an electrosurgical blade. For instance, the electrosurgical blade can include (i) a first lateral surface, (ii) a second lateral surface opposite the first lateral surface, (iii) a first major surface extending between the first lateral surface and the second lateral surface on a first side of the electrosurgical blade, and (iv) a second major surface extending between the first lateral surface and the second lateral surface on a second side of the electrosurgical blade that is opposite the first side. The first lateral surface and the second lateral surface have surface areas that are relatively small compared to surface areas of the first major surface and the second major surface such that a thickness (e.g., a dimension between the first major surface and the second major surface) of the electrosurgical blade is relatively small as compared to a length (e.g., a dimension extending between the proximal end and the distal end of the monopolar electrosurgical electrode 128) and a width (e.g., a dimension between the first latera surface and the second lateral surface).

As shown in FIG. 1, the monopolar electrosurgical electrode 128 includes at least one electrode sensor 139 embedded in at least one surface of the electrosurgical blade (e.g., embedded in the first major surface, the second lateral surface, the first lateral surface, and/or the second lateral surface). For instance, as described in greater detail below, the at least one electrode sensor 139 can be embedded between on or more electrical insulation layers on the first major surface and/or the second major surface of the electrosurgical blade.

As noted above, the electrode sensor(s) 139 can be configured to sense one or more operational conditions during an electrosurgical procedure, and transmit a sensor signal that can provide a basis for feedback control of an electrosurgical system 100 to improve the electrosurgical procedure. In one implementation, the electrode sensor(s) 139 can transmit the sensor signal to the electrosurgical tool 112, and the electrosurgical tool 112 can transmit the sensor signal to the electrosurgical generator 110. Accordingly, the electrosurgical tool 112 can include a connection mechanism for transmitting the sensor signal to the electrosurgical generator 110 (e.g., to the controller 141). The connection mechanism can be a relatively simple mechanism, such as a cable or system bus, or a relatively complex mechanism, such as a packet-based communication network (e.g., the Internet). In some instances, a connection mechanism can include a non-tangible medium (e.g., where the connection is wireless).

In some examples, responsive to the electrosurgical generator 110 receiving the sensor signal, the electrosurgical generator 110 can automatically adjust an operational parameter based on the sensor signal. In other examples, responsive to the electrosurgical generator 110 receiving the sensor signal, the electrosurgical generator 110 can generate an alert (e.g., an audible alert, a visual alert, and/or a haptic alert generated by the user interface 116) to inform an operator of the condition sensed by the electrode sensor(s) 139. Responsive to the alert, the operator can then manually adjust an operational parameter on the electrosurgical generator 110 or otherwise modify the electrosurgical procedure (e.g., by pausing, starting, and/or terminating the electrosurgical procedure using the user input device(s) 130 of the electrosurgical tool 112).

In some examples, the electrode sensor(s) 139 can include a temperature sensor. In such examples, the temperature sensor can sense a temperature of the electrosurgical blade of the monopolar electrosurgical electrode 128 before, during, and/or after an electrosurgical procedure (e.g., while cutting or coagulating tissue using the monopolar electrosurgical electrode 128). In one implementation, the temperature sensor can transmit a sensor signal that is indicative of the temperature of the monopolar electrosurgical electrode 128 to the electrosurgical generator 110 and, responsive to the sensor signal, the electrosurgical generator 110 can adjust the electrosurgical energy supplied to the electrosurgical tool 112. For instance, the electrosurgical generator 110 can adjust the electrosurgical energy supplied to the electrosurgical tool 112 by halting the supply of the electrosurgical energy, starting the supply of the electrosurgical energy, modifying a waveform of the electrosurgical energy, increasing a level of power of the electrosurgical energy, and/or decreasing a level of power of the electrosurgical energy. As described above, within examples, the electrosurgical generator 110 can adjust the electrosurgical energy (i) automatically and without user input, or (ii) responsive to user input after generating an alert via the user interface 116.

In some implementations, the electrosurgical generator 110 can perform a comparison of the temperature sensed by the temperature sensor with a threshold value. The electrosurgical generator 110 can then determine, based on the comparison, that the temperature is greater than the threshold value. Responsive to determining that the temperature is greater than the threshold value, the electrosurgical generator 110 can halt a supply of the electrosurgical energy from the electrosurgical generator to the electrosurgical tool or, in another example, decrease a power and/or adjust a waveform of the electrosurgical energy supplied to the electrosurgical tool 112. In one example, the threshold value can be less than or equal to a melting temperature of at least one of the electrical insulation layer(s). This can help to mitigate damage to the monopolar electrosurgical electrode 128. For instance, in an implementation in which the electrical insulation layer(s) include polyurethane, the threshold can be a temperature between approximately 185 degrees Celsius and approximately 225 degrees Celsius.

In another example, the threshold value can be less than a temperature at which tissue undergoes necrosis (e.g., the threshold value can be approximately 45 degrees Celsius). This can help to improve patient outcomes and/or improve electrosurgical performance. For instance, it has been found that performing electrosurgery at a relatively low-temperature is associated with a statistically significant reduction in the incidence of any type of adverse event.

In another example, the electrosurgical generator 110 can perform a comparison of the temperature sensed by the temperature sensor with a plurality of reference temperature values, where the reference temperature values are associated with a plurality of thermal effects that the target tissue may experience during the electrosurgical procedure. As an example, the reference temperatures and corresponding thermal effects can include one or more of a group consisting of: (i) first reference temperature(s) indicating that the target tissue is initially heating, (ii) second reference temperature(s) indicating that the target tissue is retracting, (iii) third reference temperature(s) indicating that the target tissue is undergoing a reduction in enzymatic activity, (iv) fourth reference temperature(s) indicating that the target tissue is experiencing protein denaturation (e.g., coagulation), (v) fifth reference temperature(s) indicating that the target tissue is losing water content (e.g., dehydrating), (vi) sixth reference temperature(s) indicating that the target tissue is experiencing cell membrane destruction and/or intercellular water ebullition, (vii) seventh reference temperature(s) indicating that the target tissue is undergoing carbonization (e.g., fulguration), (viii) eighth reference temperature(s) indicating that the target tissue is undergoing vaporization, and (ix) ninth reference temperature(s) indicating that the target tissue is burning.

In one implementation, the first reference temperature(s) can be temperatures between approximately 37 degrees Celsius and approximately 43 degrees Celsius, the second reference temperature(s) can be temperatures between approximately 43 degrees Celsius and approximately 45 degrees Celsius, the third reference temperature(s) can be temperatures above approximately 50 degrees Celsius, the fourth reference temperature(s) can be temperatures between approximately 45 degrees Celsius and approximately 60 degrees Celsius, the fifth reference temperature(s) can be temperatures between approximately 90 degrees Celsius and approximately 100 degrees Celsius, the sixth reference temperature(s) can be temperatures greater than approximately 100 degrees Celsius, the seventh reference temperature(s) can be temperatures greater than approximately 150 degrees Celsius, the eighth reference temperature(s) can be temperatures greater than approximately 300 degrees Celsius, and the ninth reference temperature(s) can be temperatures greater than approximately 500 degrees Celsius. In some examples, the reference temperatures can be based on a type of tissue for the target tissue as different types of tissue may have different demarcation points between different thermal effects.

Based on the comparison of the temperature sensed by the temperature sensor and the reference temperatures, the electrosurgical generator 110 can determine the thermal effect(s) occurring at the target tissue. Responsive to determining the thermal effect(s) occurring at the target tissue, the electrosurgical generator 110 can generate an alert (e.g., an audible alert, a visual alert, and/or a haptic alert generated by the user interface 116) to inform an operator of the thermal effect(s) determined by the electrosurgical generator 110 based on the temperature sensed by the electrode sensor(s) 139. For instance, the alert can provide an indication of the thermal effect(s) and/or provide an indication of a phase of an electrosurgical procedure in addition or in alternative to providing an indication of the temperature sensed by the electrode sensor(s) 139. In this way, the electrosurgical system 100 can provide feedback information to help inform the practioner of the status of the electrosurgical procedure. In some instances, responsive to the alert, the operator can then manually adjust an operational parameter on the electrosurgical generator 110 or otherwise modify the electrosurgical procedure (e.g., by pausing, starting, and/or terminating the electrosurgical procedure using the user input device(s) 130 of the electrosurgical tool 112).

As examples, the temperature sensor can include at least one sensor selected from a group consisting of: a thermocouple, a thermopile, a piezoelectric device, a resistive temperature measuring device, an infrared sensor, and/or a thermometer.

In some examples, the electrode sensor(s) 139 can additionally or alternatively include an electrochemical sensor. The electrochemical sensor can be configured to sense a change of an electrical potential responsive to a chemical reaction. For instance, the electrochemical sensor can include one or more electrodes that can transmit an electrical signal indicative of a presence or an amount of a chemical in contact with the electrode(s). In some implementations, the electrochemical sensor can provide for sensing at least one condition selected from among a group consisting of: (i) an impedance of tissue, (ii) a presence of cancerous tissue, (iii) a presence of dead tissue, (iv) a presence of a cancer marking dye, (v) a presence of a tissue marking dye, and (vi) a presence of alcohol.

In an implementation in which the electrochemical sensor can detect the cancer marking dye, the electrosurgical generator 110 can alert a user that a predetermined amount of cancerous or marked tissue has been removed. For instance, the electrochemical sensor can be configured to detect an electrochemical signature of the cancer marking dye. In one implementation, the electrochemical sensor can include a micro hot-plate solid state electrochemical sensor. Additionally, in some implementations, the monopolar electrosurgical electrode 128 can include a gas permeable membrane adjacent to the electrochemical sensor. In this arrangement, the gas permeable membrane can permit a gas to pass through an exterior surface of the monopolar electrosurgical electrode to facilitate sensing the chemical of interest (e.g., the cancer marking dye). As examples, the gas permeable membrane can include a porous polytetrafluoroethylene (PTFE), carbon nanotubes, and/or an inorganic semiconductor material. Among other benefits, the electrochemical sensor that can detect the cancer marking dye help to reduce or obviate a need for a neoprobe in some instances.

As noted above, in some implementations, the electrochemical sensor can sense a presence of alcohol. For instance, responsive to the electrochemical sensor detecting the presence of alcohol, the electrosurgical generator 110 can halt and/or prevent the supply of the electrosurgical energy from the electrosurgical generator 110 to the electrosurgical tool 112. This can help to improve safety by mitigating a risk of fire during an electrosurgical procedure.

In an implementation in which the electrochemical sensor can sense the impedance of the tissue, the electrosurgical generator 110 can use the impedance as a basis for controlling the supply of the electrosurgical energy. For instance, in one implementation, the electrosurgical generator 110 can store a plurality of power values and a plurality of impedance values, where each power value corresponds to a respective one of the impedance values. The power value can relate to a desired power of the output power for a measured impedance of the target tissue, which can achieve a desired clinical outcome during electrosurgery. In this arrangement, the electrosurgical generator 110 can use the impedance indicated by the sensor signal to identify the power value that corresponds to the impedance, and responsively provide the electrosurgical energy with the identified power value.

As an example, the electrochemical sensor can include a plurality of electrodes. For instance, the electrochemical sensor can include a working electrode and at least one of a counter electrode or a reference electrode. The electrochemical sensor can further include a reagent. The reagent can be selected based on a target chemical for which the electrochemical sensor is configured to detect. As examples, the reagent can include at least one reagent selected from a group consisting of: fluorescence, antigens, methylene blue, 5-aminolevulinic acid (5-ALA), cyanine-based dyes, including indocyanine green (ICG), IRDye800, Dyomics dyes, and quantum dots.

In some examples, the electrode sensor(s) 139 can additionally or alternatively include a force sensor. The force sensor can help to avoid damage to the monopolar electrosurgical electrode 128 due to excessive forces applied to the monopolar electrosurgical electrode 128 before, during, or after an electrosurgical procedure. Accordingly, the force sensor can be configured to sense a force applied to the monopolar electrosurgical electrode 128. As examples, the force sensor can include at least one sensor selected from a group consisting of: a pressure sensor, a force plate, and a strain gage.

In one implementation, the electrosurgical generator 110 can receive the sensor signal indicating a force sensed by the force sensor. The electrosurgical generator 110 can then perform a comparison of the force sensed by the force sensor to a threshold force and, based on the comparison, determine that the force sensed by the force sensor is greater than the threshold force. Responsive to determining that the force sensed by the force sensor is greater than the threshold force, the electrosurgical generator 110 can cause the user interface 116 to provide an alarm (e.g., a visual alert, an audible alert, and/or a haptic alert) to indicate to the user that an excessive amount of force has been applied to the monopolar electrosurgical electrode 128. The user may then inspect the monopolar electrosurgical electrode 128, replace the monopolar electrosurgical electrode 128 on the electrosurgical tool 112, and/or replace the entire electrosurgical tool 112. In another implementation, the electrosurgical generator 110 can additionally or alternatively halt or prevent supplying the monopolar electrosurgical electrode 128 until the user clears a fault condition (e.g., by providing an input using the user interface 116 and/or replacing the electrosurgical tool 112).

In some examples, the electrode sensor(s) 139 can additionally or alternatively include a mass loading sensor. The mass loading sensor can be configured to sense a mass of a load on the monopolar electrosurgical electrode 128. By sensing the mass of the load on the monopolar electrosurgical electrode 128, the mass loading sensor can provide an indication of an amount of tissue adhesion and/or char buildup on the monopolar electrosurgical electrode 128. This can help to reduce (or prevent) excessive build-up of tissue and/or eschar on the monopolar electrosurgical electrode 128 and thereby improve cutting and/or coagulation performance. As examples, the mass loading sensor can include at least one sensor selected from among a group consisting of: a resonant frequency detector device (e.g., quartz crystal microbalance, a mechanical resonator, a cantilever device, and/or a piezoelectric device) and a device that can detect a change in capacitance due to mass/area loading (e.g., a parallel plate capacitor, and/or an interdigitated, suspended plate).

In one implementation, the electrosurgical generator 110 can receive the sensor signal indicating the mass sensed by the mass loading sensor. The electrosurgical generator 110 can then perform a comparison of the mass sensed by the mass loading sensor to a threshold mass and, based on the comparison, determine that the mass sensed by the mass loading sensor is greater than the threshold mass. Responsive to determining that the mass sensed by the mass loading sensor is greater than the threshold mass, the electrosurgical generator 110 can cause the user interface 116 to provide an alarm (e.g., a visual alert, an audible alert, and/or a haptic alert) to indicate to the user that an excessive amount of the load is present on the monopolar electrosurgical electrode 128. The user may then inspect the monopolar electrosurgical electrode 128, replace the monopolar electrosurgical electrode 128 on the electrosurgical tool 112, and/or replace the entire electrosurgical tool 112. In another implementation, the electrosurgical generator 110 can additionally or alternatively halt or prevent supplying the monopolar electrosurgical electrode 128 until the user clears a fault condition (e.g., by providing an input using the user interface 116 and/or replacing the electrosurgical tool 112).

In some implementations, the electrode sensor(s) 139 can additionally or alternatively include a dielectric sensor. The dielectric sensor can be configured to sense a dielectric property of the monopolar electrosurgical electrode 128 such as, for instance, a change in the dielectric property due to a change in an enamel and/or the electrical insulation layer(s) of the monopolar electrosurgical electrode 128. Additionally or alternatively, the dielectric sensor can be configured to sense a change in a dielectric property due to, for example, a build-up of eschar on a surface of the monopolar electrosurgical electrode 128. In some examples, the electrosurgical generator 110 can receive the sensor signal indicating the dielectric property sensed by the dielectric sensor and determine, based on the sensor signal, the dielectric property has changed in a manner that is indicative of a change to a structural integrity of the monopolar electrosurgical electrode 128 and/or a build-up of eschar. Responsive to determining that the dielectric property changed, the electrosurgical generator 110 can cause the user interface 116 to provide an alarm (e.g., a visual alert, an audible alert, and/or a haptic alert) to indicate to the user that the monopolar electrosurgical electrode 128 and/or the electrosurgical tool 112 should be replaced and/or cleaned. The user may then inspect the monopolar electrosurgical electrode 128, replace the monopolar electrosurgical electrode 128 on the electrosurgical tool 112, replace the entire electrosurgical tool 112, and/or clean the monopolar electrosurgical electrode 128. In another implementation, the electrosurgical generator 110 can additionally or alternatively halt or prevent supplying the monopolar electrosurgical electrode 128 until the user clears a fault condition (e.g., by providing an input using the user interface 116, replacing the electrosurgical tool 112, replacing the monopolar electrosurgical electrode 128, and/or cleaning the monopolar electrosurgical electrode 128).

As examples, the dielectric sensor can include one or more sensors selected from a group consisting of: a resistive sensor and a capacitive sensor. In one implementation of a resistive sensor, the dielectric sensor can detect an increase in leakage current responsive to the change in the dielectric property. In one implementation, the dielectric sensor can include a three-dimensional (3D) interdigitated capacitor with a conformal insulation coating covering the 3D interdigitated capacitor. In this implementation, a degradation in the electrode insulation (e.g., pin hole defects, flaking, voids, and/or fissures) can be detected due a change in a dielectric constant of the conformal insulation coating.

In an implementation in which the dielectric sensor is covered by a conformal insulation coating, the dielectric property decreases when a sufficiently large void forms in the conformal insulation coating. In this implementation, the electrosurgical generator 110 can receive the sensor signal indicating the dielectric property sensed by the dielectric sensor. The electrosurgical generator 110 can then perform a comparison of the dielectric property sensed by the dielectric sensor to a threshold dielectric value and, based on the comparison, determine that the dielectric property sensed by the dielectric sensor is less than the threshold dielectric value. Responsive to determining that the dielectric property sensed by the dielectric sensor is less than the threshold dielectric value, the electrosurgical generator 110 can cause the user interface 116 to provide an alarm (e.g., a visual alert, an audible alert, and/or a haptic alert) to indicate to the user that the monopolar electrosurgical electrode 128 and/or the electrosurgical tool 112 should be replaced. The user may then inspect the monopolar electrosurgical electrode 128, replace the monopolar electrosurgical electrode 128 on the electrosurgical tool 112, and/or replace the entire electrosurgical tool 112. In another implementation, the electrosurgical generator 110 can additionally or alternatively halt or prevent supplying the monopolar electrosurgical electrode 128 until the user clears a fault condition (e.g., by providing an input using the user interface 116 and/or replacing the electrosurgical tool 112).

In another implementation, the dielectric sensor can include a plurality of spatial separated RF isolated interdigitated electrode arrays that are exposed on the surface of the monopolar electrosurgical electrode 128. In this arrangement, the dielectric sensor can initially sense a dielectric property associated with air adjacent to the dielectric sensor. During the electrosurgical procedure, the dielectric sensor can initially sense a decrease in the dielectric constant relative to the dielectric constant of air due to cutting of tissue resulting in a steam and/or vapor envelope at the surface of the monopolar electrosurgical electrode 128. As the electrosurgical procedure progresses, eschar may build up on the surface of the monopolar electrosurgical electrode 128. The dielectric sensor can sense the eschar by sensing an increase in the dielectric constant as the eschar builds up. The electrosurgical generator 110 can receive and process the sensor signal based on a threshold analysis and/or signature detection analysis to determine when a threshold amount of eschar has built-up, and responsively perform one or more of the functions described above (e.g., generating an alert and/or adjusting a supply of the electrosurgical energy).

In some examples, the electrode sensor(s) can additionally or alternatively include a conductivity sensor. The conductivity sensor can be configured to sense a conductivity of tissue. For instance, similar to some implementations of the electrochemical sensor described above, the conductivity sensor can provide for sensing an impedance of the tissue. In an implementation in which the conductivity sensor can sense the impedance of the tissue, the electrosurgical generator 110 can use the impedance as a basis for controlling the supply of the electrosurgical energy. For instance, in one implementation, the electrosurgical generator 110 can store a plurality of power values and a plurality of impedance values, where each power value corresponds to a respective one of the impedance values. The power value can relate to a desired power of the output power for a measured impedance of the target tissue, which can achieve a desired clinical outcome during electrosurgery. In this arrangement, the electrosurgical generator 110 can use the impedance indicated by the sensor signal to identify the power value that corresponds to the impedance, and responsively provide the electrosurgical energy with the identified power value. As examples, the conductivity sensor can include at least one sensor selected from a group consisting of: a contacting conductive sensor and an inductive conductive sensor.

In some examples, the electrode sensor(s) 139 can additionally or alternatively include a metal detector sensor. In general, the metal detector sensor is configure dot detect the presence of a metal within a proximity of the monopolar electrosurgical electrode 128. As one example, the metal detector sensor can include an oscillator that can produce an alternating current that passes through a coil producing an alternating magnetic field. When a piece of electrically conductive metal is close to the coil, eddy currents will be induced (inductive sensor) in the metal, and this produces a magnetic field of its own. When another coil is used to measure the magnetic field (acting as a magnetometer), the change in the magnetic field due to the metallic object can be detected.

In one implementation, responsive to the sensor signal indicating that a piece of metal is within predetermined distance of the monopolar electrosurgical electrode 128, the electrosurgical generator 110 can halt the supply of the electrosurgical energy to the electrosurgical tool 112. In another implementation, responsive to the sensor signal indicating that a piece of metal is within predetermined distance of the monopolar electrosurgical electrode 128, the electrosurgical generator 110 can cause the user interface 116 to provide an alarm (e.g., a visual alert, an audible alert, and/or a haptic alert) to indicate to the user that the monopolar electrosurgical electrode 128 is near the piece of metal. These implementations can help to mitigate a risk of arcing between the monopolar electrosurgical electrode 128 and the piece of metal (e.g., a metal implant or device).

In some examples, the electrode sensor(s) 139 can additionally or alternatively include one or more tracking sensors that can sense at least one of: a location of the monopolar electrosurgical electrode 128 and an orientation of the monopolar electrosurgical electrode 128. For instance, the electrode sensor(s) 139 can include one or more electromagnetic field sensors (e.g., one or more electromagnetic coils) that can sense an electromagnetic field generated by an electromagnetic field generator (e.g., via an inductive coupling) and transmit sensor signal that is indicative of a strength of the electromagnetic field. The electrode sensor(s) 139 can additionally or alternatively include an inertial measurement sensor that can transmit a sensor signal that is indicative of a sensed inertia of the monopolar electrosurgical electrode 128.

In one implementation, the electrosurgical generator 110 can determine based on the sensor signal that the monopolar electrosurgical electrode 128 is located outside of a surgical area. Response to determining that the monopolar electrosurgical electrode 128 is located outside of a surgical area, the electrosurgical generator 110 can halt a supply of the electrosurgical energy and/or generate an alert via the user interface 116. This can help to improve safety by mitigating (or preventing) inadvertent activation of the electrosurgical tool 112 when the electrosurgical tool 112 is located outside of the surgical site.

To help track the location and/or the orientation of the monopolar electrosurgical electrode 128 relative to the surgical site, the electrosurgical system 100 can further include one or more reference tracking sensors coupled to a patient at the surgical site. In this arrangement, the electrosurgical generator 110 can compare sensor signals received from the tracking sensor(s) on the monopolar electrosurgical electrode 128 to sensor signals received from the reference tracking sensor(s) to determine whether the monopolar electrosurgical electrode 128 is located outside of a surgical area.

In some examples, the electrode sensor(s) 139 can include a light sensor. For instance, the light sensor can include a photoresistor, a photodiode, and/or a phototransistor. In general, the light sensor can sense an amount of light at the monopolar electrosurgical electrode 128 and generate a sensor signal indicative of the sensed amount of light. In one implementation, the electrosurgical system 100 can adjust, based on the amount of light indicated by the sensor signal, an amount of light that is output by the light source 140.

For instance, in one implementation, the electrosurgical generator 110 can store a plurality of lumen output values and a plurality of light measurement values, where each lumen output value corresponds to a respective one of the light measurement values. The lumen output value can relate to a desired lumen output of the light source 140 for a light measurement value such that a desired light condition can be achieved at the surgical site. In this arrangement, the electrosurgical generator 110 can use the light measurement value indicated by the sensor signal to identify the lumen output value that corresponds to the light measurement value, and responsively cause the light source 140 to generate light with the corresponding lumen output value. This can help to improve visualization of the surgical site, conserve power, and/or extend battery life.

In some examples, the electrode sensor(s) 139 can include a smoke detector sensor. For instance, the light sensor can include a photoelectric detector and/or an ionization detector. Additionally or alternatively, the smoke detector sensor can include the electrochemical sensor(s) described above (e.g., including a gas permeable membrane that permits smoke to pass through the surface of the monopolar electrosurgical electrode 128 to the electrochemical sensor). In general, the smoke detector can sense an amount of smoke at the monopolar electrosurgical electrode 128 and generate a sensor signal indicative of the sensed amount of smoke. In one implementation, the electrosurgical system 100 can adjust, based on the amount of smoke indicated by the sensor signal, an amount of suction that is provided by the suction pump 146.

For instance, in one implementation, the electrosurgical generator 110 can store a plurality of suction force values and a plurality of smoke measurement values, where each suction force value corresponds to a respective one of the smoke measurement values. Suction force value can relate to a desired amount of suction to be provided at the smoke inlet to the smoke evacuation channel 148 for a given smoke measurement value such surgical smoke can be efficiently captured at the surgical site. In this arrangement, the electrosurgical generator 110 can use the smoke measurement value indicated by the sensor signal to identify the suction force value that corresponds to the smoke measurement value, and responsively cause the suction pump 146 to generate suction with the corresponding suction force value. This can help to mitigate surgical smoke at a surgical site.

In some examples, the electrode sensor(s) 139 can additionally or alternatively include a sensor that can provide for determining information relating to the monopolar electrosurgical electrode 128 that is coupled to the electrosurgical tool 112 and/or the electrosurgical generator 110. For example, the electrode sensor(s) 139 can include one or more resistive elements having a predefined resistance. The electrosurgical generator 110 and/or the electrosurgical tool 112 can be configured to apply an electrical potential to the resistive element(s) to determine the resistance of the resistive element(s), and determine, based on the determined resistance, at least one item of information selected from a group including: (i) a type of the monopolar electrosurgical electrode 128, (ii) a size of the monopolar electrosurgical electrode 128, (iii) a shape of the monopolar electrosurgical electrode 128, (iv) a manufacturer of monopolar electrosurgical electrode 128, and (v) a number of times the monopolar electrosurgical electrode 128 has used.

The electrosurgical tool 112 and/or the electrosurgical generator 110 can be configured to couple and decouple with a plurality of monopolar electrosurgical electrodes 128, where the contact(s) of at least one of the monopolar electrosurgical electrodes 128 have a different resistance than at least another one of the monopolar electrosurgical electrodes 128. In one implementation, the electrosurgical generator 110 can adjust an operational parameter (e.g., a power of the electrosurgical energy, a waveform of the electrosurgical energy, and/or a duration for application of the electrosurgical energy) based on the information determined for the monopolar electrosurgical electrode 128. In another implementation, the electrosurgical generator 110 can additionally or alternatively generate an alert (e.g., an audible alert, a visual alert, and/or a haptic alert generated by the user interface 116) to communicate the information determined for the monopolar electrosurgical electrode 128. Responsive to the alert, the operator can then manually adjust an operational parameter on the electrosurgical generator 110 or otherwise modify the electrosurgical procedure (e.g., by pausing, starting, and/or terminating the electrosurgical procedure using the user input device(s) 130 of the electrosurgical tool 112). These implementations can help to tailor operational parameters to the specific monopolar electrosurgical electrode 128 and/or improve operational safety. Additionally, in an implementation in which the item of information determined from the resistive element(s) include the number of times the monopolar electrosurgical electrode 128 has been used, the resistive element(s) can help to prevent reprocessing of the monopolar electrosurgical electrode 128.

In one implementation, the resistive element(s) can have a single resistance value and one or more items of information can be determined from the single resistance value. In another implementation, the resistive element(s) include a plurality of resistive elements that each have a respective resistance value. In this implementation, different items of information can be determined from different ones of the resistive elements.

In another implementation, the resistive elements can include a plurality of resistance contacts. In an example, a first one of the resistive elements can provide for determining first information relating to the monopolar electrosurgical electrode 128, and a second one of the resistive elements can provide for determining second information relating to the monopolar electrosurgical electrode 128.

As noted above, the monopolar electrosurgical electrode 128 includes at least one electrode sensor 139 embedded in at least one surface of the electrosurgical blade (e.g., embedded in the first major surface, the second lateral surface, the first lateral surface, and/or the second lateral surface). In some examples, the monopolar electrosurgical electrode 128 can include a first electrode sensor embedded between one or more electrical insulation layers on the first major surface of the electrosurgical blade, and a second electrode sensor embedded between the electrical insulation layer(s) on the second major surface of the electrosurgical blade. The first electrode sensor and the second electrode sensor can be different types of electrode sensors. For instance, the first electrode sensor and the second electrode sensor can be different sensors respectively selected from a group consisting of: a temperature sensor, an electrochemical sensor, a force sensor, a mass loading sensor, a dielectric sensor, a conductivity sensor, a metal detector sensor, a tracking sensor, a light sensor, and a smoke sensor.

In other examples, the monopolar electrosurgical electrode 128 can include only the first electrode sensor embedded in only one surface of the monopolar electrosurgical electrode 128. In such examples, the monopolar electrosurgical electrode 128 can omit the second electrode sensor described above. As described above, the first electrode sensor can be selected from the group consisting of: a temperature sensor, an electrochemical sensor, a force sensor, a mass loading sensor, a dielectric sensor, a conductivity sensor, a metal detector sensor, a tracking sensor, a light sensor, and a smoke sensor. In still other examples, the monopolar electrosurgical electrode 128 can include more than two electrode sensors 139.

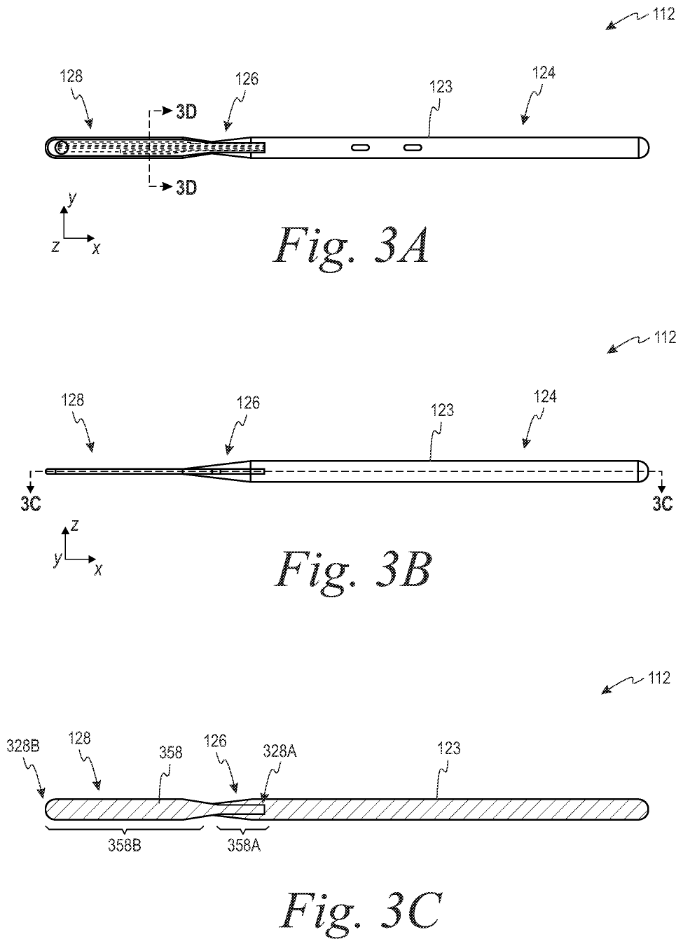

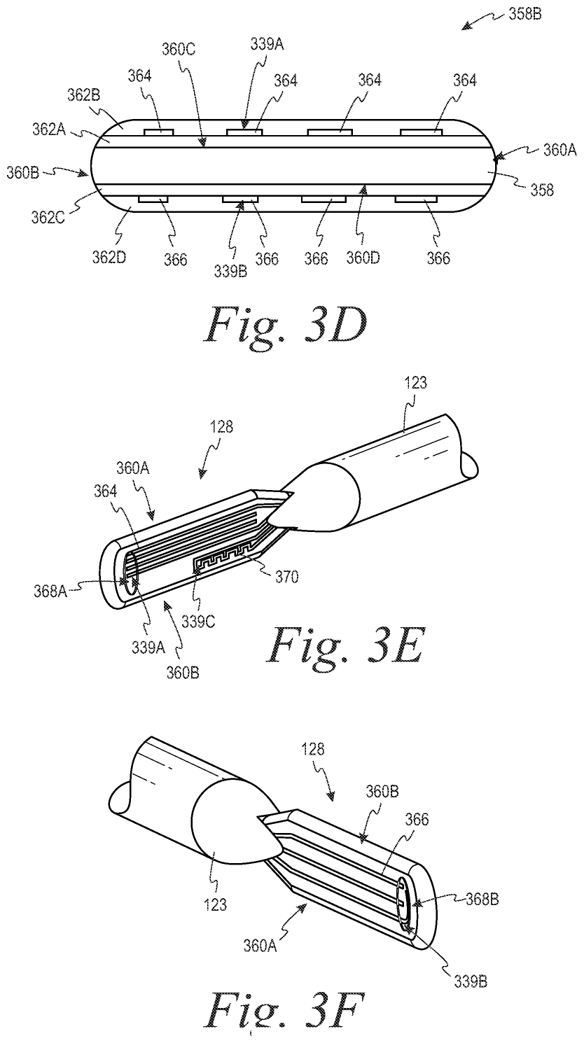

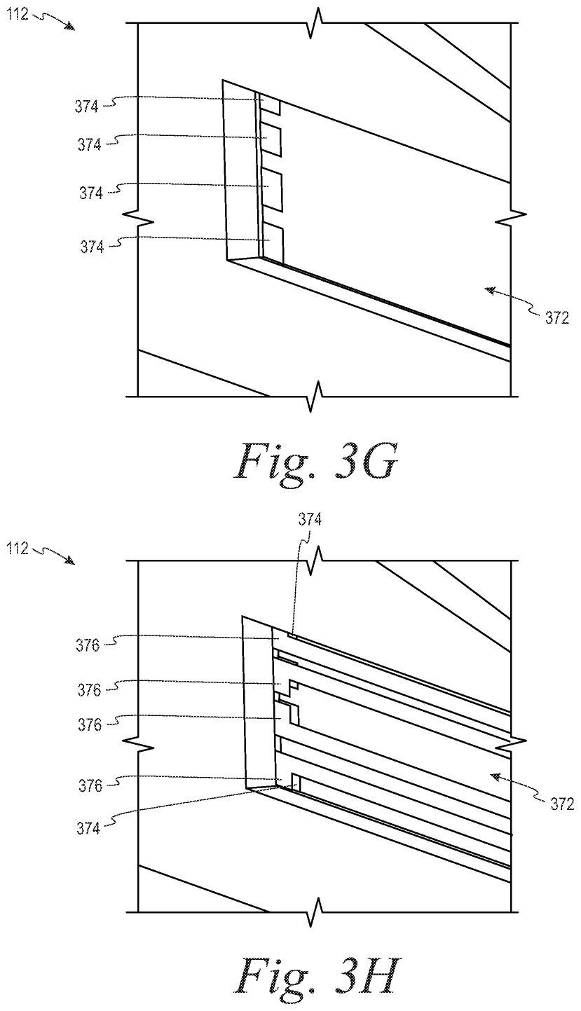

Referring now to FIGS. 3A-3H, an implementation of the electrosurgical tool 112 and the monopolar electrosurgical electrode 128 is shown according to an example. In particular, FIG. 3A depicts a first side view of the electrosurgical tool 112, FIG. 3B depicts a second side view of the electrosurgical tool 112, FIG. 3C depicts a cross-sectional view of the electrosurgical tool 112 taken through line 3C in FIG. 3B, FIG. 3D depicts a cross-sectional view of the monopolar electrosurgical electrode 128 taken through line 3D in FIG. 3A, FIG. 3E depicts a first perspective view of the monopolar electrosurgical electrode 128, FIG. 3F depicts a second perspective view of the monopolar electrosurgical electrode, FIG. 3G depicts a receptacle including one or more receptacle contacts for coupling with one or more electrode contacts on a shank portion of the monopolar electrosurgical electrode, and FIG. 3H depicts the sensor contacts of the shank portion of the monopolar electrosurgical electrode electrically coupled to the receptacle contacts of the receptacle according to the example.

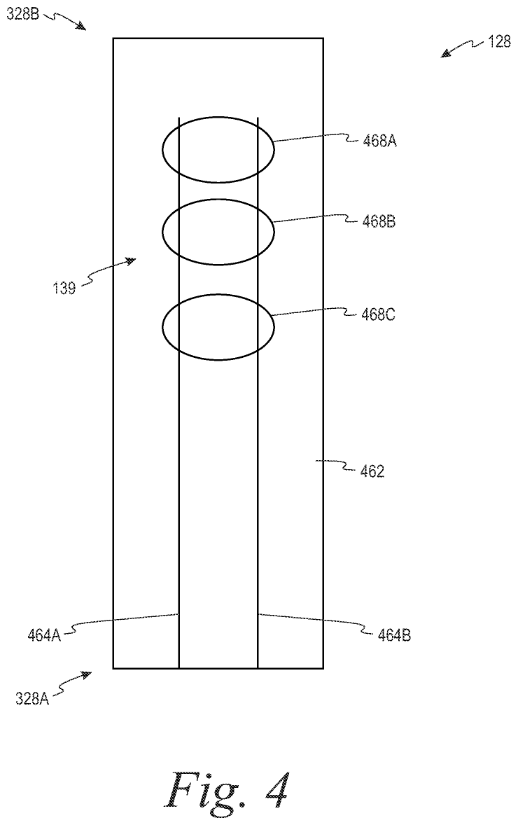

As shown in FIGS. 3A-3B, the electrosurgical tool 112 includes the housing 123 and the monopolar electrosurgical electrode 128 extending from a distal end of the housing 123. In this example, the housing 123 is configured such that the shaft 126 is not movable or rotatable relative to the handle 124 of the housing 123. However, as described above with respect to FIG. 1 and as described below with respect to FIG. 4, the shaft 126 can be axially movable and/or rotatable relative to the handle 124 in other examples.

Additionally, as shown in FIG. 3A, the user input device(s) 130 include a first button 330A and a second button 330B on an exterior surface of the housing 123. In one implementation, the first button 330A can be actuated to operate the electrosurgical tool 112 in a cutting mode of operation, and the second button 330B can be actuated to operate the electrosurgical tool 112 in a coagulation mode of operation. As described above, the user input device(s) 130 can be configured differently in other examples. For instance, the electrosurgical tool 112 can be operable in a lesser quantity of modes of operation, a greater quantity of modes of operation, and/or different types of modes of operation in other examples (e.g., such as the example modes of operation described above). Additionally, for instance, the at least one user input device 130 can additionally or alternatively include the user interface 116 of the electrosurgical generator 110 and/or another external device (e.g., a footswitch) for operating the electrosurgical tool 112 in one or more modes of operation.

The implementation of the electrosurgical tool 112 shown in FIGS. 3A-3H omits the light source 140, the optical structure 142, the smoke evacuation channel 148, the smoke evacuation chamber 152, and the smoke tube 150. However, as described above with respect to FIG. 1, the electrosurgical tool 112 can include additional or alternative features such as, for instance, the light source 140, the optical structure 142, the smoke evacuation channel 148, the smoke evacuation chamber 152, and the smoke tube 150 in other examples. Also, in FIGS. 3A-3B, the power cord 122 is not depicted for ease of illustration purposes only.

As shown in FIG. 3C, the monopolar electrosurgical electrode 128 includes an electrosurgical substrate 358 formed from an electrically conductive material. As an example, the electrically conductive material can be stainless steel. The electrosurgical substrate 358 can extend in an axial direction from a proximal end 328A of the monopolar electrosurgical electrode 128 to a distal end 328B of the monopolar electrosurgical electrode 128. The electrosurgical substrate 358 can further include a shank portion 358A that can receive the electrosurgical energy from the electrosurgical tool 112 (e.g., via the housing conductor 134 and the shaft conductor 136 as described above), and an electrosurgical blade 358B that is configured for at least one of cutting or coagulation of tissue by the electrosurgical energy received from the electrosurgical tool 112.

The shank portion 358A is proximal of the electrosurgical blade 358B. Additionally, the shank portion 358A can be received in the housing 123 and electrically coupled to the shaft conductor 136 and/or the housing conductor 134. For example, in one implementation, the electrosurgical tool 112 can include a receptacle that can receive and electrically couple the shank portion 358A of the monopolar electrosurgical electrode 128 to the electrosurgical tool 112. As an example, the receptacle and the shank portion 358A of the monopolar electrosurgical electrode 128 can be configured to couple to each other by friction-fit. Accordingly, the receptacle and the shank portion 358A can have respective sizes and/or respective shapes that provide for a friction-fit coupling between the receptacle and the shank portion 358A when the shank portion 358A is inserted in the receptacle. This can allow for the monopolar electrosurgical electrode 128 to be releasably coupled to the electrosurgical tool 112, which can facilitate an interchangeability of a plurality of the monopolar electrosurgical electrodes 128 with the electrosurgical tool 112. The receptacle and the shank portion 358A can be mechanically keyed to ensure the correct electrical connections are made. In other examples, the shank portion 358A can be coupled to the receptacle by another type of releasable coupling (e.g., a threaded coupling) or a non-releasable coupling (e.g., via welding and/or soldering).

Within examples, the receptacle can also include a conductor that can electrically couple the shank portion 358A of the monopolar electrosurgical electrode 128 to the electrosurgical energy supplied to the electrosurgical tool 112 by the electrosurgical generator 110. For instance, the receptacle 137 can be electrically coupled to the shaft conductor 136 and/or the housing conductor 134 (e.g., by a conductive material).

As noted above, the electrosurgical blade 358B can be configured for at least one of cutting, coagulating, ablating, or sealing of tissue by the electrosurgical energy received from the electrosurgical tool 112. As shown in FIG. 3C, the electrosurgical blade 358B provides a working portion of the monopolar electrosurgical electrode 128 that extends distally of the shank portion 358A toward the distal end 328B. In this arrangement, the monopolar electrosurgical electrode 128 can receive the electrosurgical energy from the electrosurgical tool 112 at the shank portion 358A, transmit the electrosurgical energy distally along the electrosurgical substrate 358 from the shank portion 358A to the electrosurgical blade 358B, and apply the electrosurgical energy from the electrosurgical blade 358B to the target tissue to perform an electrosurgical procedure (e.g., cutting, coagulating, ablating, and/or sealing the target tissue).

In form of the electrosurgical blade 358B, the working portion of the monopolar electrosurgical electrode 128 can be configured to focus the electrosurgical energy at one or more lateral surfaces to enhance the cutting and/or coagulating performance of the monopolar electrosurgical electrode 128 as compared to non-blade shaped electrodes (e.g., catheter type electrodes). For instance, as shown in FIG. 3D, the electrosurgical blade 358B can include (i) a first lateral surface 360A, (ii) a second lateral surface 360B opposite the first lateral surface 360A, (iii) a first major surface 360C extending between the first lateral surface 360A and the second lateral surface 360B on a first side of the electrosurgical blade 358B, and (iv) a second major surface 360D extending between the first lateral surface 360A and the second lateral surface 360B on a second side of the electrosurgical blade 358B that is opposite the first side. The first lateral surface 360A and the second lateral surface 360B have surface areas that are relatively small compared to surface areas of the first major surface 360C and the second major surface 360D such that the thickness (e.g., a dimension along a z-axis in FIGS. 3A-3B between the first major surface 360C and the second major surface 360D) of the electrosurgical blade 358B is relatively small as compared to a length (e.g., a dimension along an x-axis in FIGS. 3A-3B extending between the proximal end 328A and the distal end 28B of the monopolar electrosurgical electrode 128) and a width (e.g., a dimension along a y-axis between the first lateral surface 360A and the second lateral surface 360B). Additionally, in FIGS. 3A-3D, the length of the electrosurgical blade 358B can be greater than the width of the electrosurgical blade 358B, and the width of the electrosurgical blade 358B can be greater than the thickness of the electrosurgical blade 358B.

In the example shown in FIGS. 3A-3D, the first major surface 360C and the second major surface 360D are substantially planar and parallel to each other. In other examples, the first major surface 360C and the second major surface 360D can have a different shape and/or configuration. For instance, in another example, the first major surface 360C and the second major surface 360D can have at least one feature selected from among: (i) a curved shape, (ii) a convex shape, (iii) a tapering shape such that the thickness of the electrosurgical blade 358B becomes smaller in a direction from a center of the first major surface 360C and/or the second major surface 360D toward the first lateral surface 360A and/or the second lateral surface 360B, (iv) a shape of the first major surface 360C can be an inverse of a shape of the second major surface 360D, and (v) a shape of the first major surface 360C may not be an inverse of a shape of the second major surface 360D.

Also, in the example shown in FIGS. 3A-3D, the first lateral surface 360A and the second lateral surface 360B are curved and have the same thickness between the first major surface 360C and the second major surface 360D. In other examples, the first lateral surface 360A and the second lateral surface 360B can have a different shape and/or configuration. For instance, in another example, the first lateral surface 360A and/or the second lateral surface 360B can have at least one feature selected from among: (i) a planar shape, (ii) a tapering shape such that the width of the electrosurgical blade 358B becomes greater in a direction from a center of the first lateral surface 360A and/or the second lateral surface 360B toward the first major surface 360C and/or the second major surface 360D, (iii) a shape of the first lateral surface 360A can be an inverse of a shape of the second lateral surface 360B, and (iv) a shape of the first major surface 360C may not be an inverse of a shape of the second major surface 360D.

As described above, the monopolar electrosurgical electrode 128 includes the at least one electrode sensor 139 embedded in at least one surface of the electrosurgical blade (e.g., embedded in the first lateral surface 360A, the second lateral surface 360B, the first major surface 360C, and/or the second major surface 360D). In the example shown in FIGS. 3D-3F, the electrode sensor(s) 139 includes a first electrode sensor 339A embedded between a plurality of electrical insulation layers 362A-362B on the first major surface 360C, and a second electrode sensor 339B embedded between a plurality of electrical insulation layers 362C-362D on the second major surface 360D.

The first electrode sensor 339A and the second electrode sensor 339B can be any type sensors described above for the electrode sensor(s) 139. Accordingly, the first electrode sensor 339A can be a sensor selected from among a group of sensors consisting of: (i) a temperature sensor, (ii) an electrochemical sensor, (iii) a force sensor, (iv) a mass loading sensor, (v) a dielectric sensor, (vi) a conductivity sensor, (vii) a metal detector sensor, (viii) a tracking sensor configured to sense at least one of: a location of the monopolar electrosurgical electrode and an orientation of the monopolar electrosurgical electrode, (ix) light sensor, and (x) a smoke detector sensor. Similarly, the second electrode sensor 339B can be a sensor selected from among a group of sensors consisting of: (i) a temperature sensor, (ii) an electrochemical sensor, (iii) a force sensor, (iv) a mass loading sensor, (v) a dielectric sensor, (vi) a conductivity sensor, (vii) a metal detector sensor, (viii) a tracking sensor configured to sense at least one of: a location of the monopolar electrosurgical electrode and an orientation of the monopolar electrosurgical electrode, (ix) light sensor, and (x) a smoke detector sensor.

In some examples, the first electrode sensor 339A and the second electrode sensor 339B can be the same type of electrode sensor 139 (e.g., the first electrode sensor 339A and the second electrode sensor 339B can both be temperature sensors). This can help to enhance an accuracy of the sensed information relating to the operating conditions and/or provide redundancy in sensing the operating conditions. In other examples, the first electrode sensor 339A can be a first type of electrode sensor 139 and the second electrode sensor 339B can be a second type of electrode sensor 139, where the first type and the second type are different from each other. This can help to sense a plurality of different operating conditions for providing feedback and/or controlling the electrosurgical operation.

The first electrode sensor 339A and/or the second electrode sensor 339B can each include one or more electrically conductive element embedded between the electrical insulation layers 362A-362D, respectively. The electrically conductive elements can be configured for sensing the operating conditions described above, and/or communicating the sensor signals from the electrode sensors 139 to the electrosurgical tool 112 and/or the electrosurgical generator 110. As described in further detail below, the electrically conductive element(s) of the first electrode sensor 339A and/or the second electrode sensor 339B can be formed by a process including lithography patterning and/or metal lift off of at least a portion of an electrically conductive material. Additionally or alternatively, the electrically conductive element(s) can be formed by a process that includes micro-transfer printing, ebeam lithography, nanoimprinting lithography, micro-contact printing lithography, focus ion beam lithography, deep ultraviolet (UV) lithography, x-ray lithography, LIGA, wet chemistry (wet etching), ion milling, reactive ion etching), atomic layer deposition (e.g., selective area and/or non-selective area), chemical vapor deposition, and/or physical vapor deposition.

The electrical insulation layers 362A-362D can help to inhibit (or prevent) electromagnetic interference from affecting measurements by the electrode sensor(s) 139 and/or the sensor signals transmitted by the electrode sensor(s) 139 (e.g., the first electrode sensor 339A and/or the second electrode sensor 339B). For example, the electrical insulation layers 362A, 362C can help to inhibit (or prevent) the flow of electric current between the first major surface 360C and the first electrode sensor 339A, and the flow of electric current between the second major surface 360D and the second electrode sensor 339B while the electrosurgical energy flows through the electrosurgical substrate 358. Additionally, for example, the electrical insulation layers 362B, 362D can help to inhibit or prevent at least a portion (or an entirety) of the first electrode sensor 339A and/or the second electrode sensor 339B from contacting substances (e.g., blood, irrigation fluids, tissue, and/or char). The electrical insulation layers 362A-362D can additionally or alternatively help to focus the electrosurgical energy at the first lateral surface 360A and/or the second lateral surface 360B to apply the electrosurgical energy to the target tissue with reduced spread to non-target tissue (e.g., relative to an electrode that applies greater electrosurgical energy through the first and second major surfaces).

As examples, the electrical insulation layers 362A-362D can be formed from one or more materials selected from a group of materials consisting of: aluminum oxide (Al₂O₃), silicon dioxide (SiO₂), silicon nitride (Si₃N₄), silicon oxynitride (SiON), and tantalum pentoxide (Ta₂O₅). In some implementations, the electrical insulation layers 362A-362D can be passivation layers formed from a passivation process. In some examples, when the electrical insulation layers 362A-362D are formed from the passivation process, the electrical insulation layers 362A can be more resistant to environmental conditions experienced during electrosurgery than when the electrical insulation layers 362A-362D are formed by other manufacturing processes. In other implementations, the electrical insulation layers 362A-362D can be additionally or alternatively formed by at least one process selected from among a group consisting of: physical vapor deposition, chemical vapor deposition, printing, spin coating, spray coating, sintering, and thermal coating.

In FIGS. 3D-3F, at least a portion of the first electrode sensor 339A and the second electrode sensor 339B are each embedded between a respective pair of the electrical insulation layers 362A-362D. For instance, as shown in FIG. 3D, the electrical insulation layers 362A-362D can include a first electrical insulation layer 362A, a second electrical insulation layer 362B, a third electrical insulation layer 362C, and a fourth electrical insulation layer 362D. As shown in the example depicted in FIGS. 3D-3E, the first electrical insulation layer 362A covers the first major surface 360C of the electrosurgical blade 358B, the first electrode sensor 339A includes a plurality of conductive traces 364 on the first electrical insulation layer 362A, and the second electrical insulation layer 362B covers the conductive traces 364 of the first electrode sensor 339A and the first electrical insulation layer 362A. Similarly, as shown in FIG. 3D and FIG. 3F, the third electrical insulation layer 362C covers the second major surface 360D of the electrosurgical blade 358B, the second electrode sensor 339B includes a plurality of conductive traces 366 on the third electrical insulation layer 362C, and the fourth electrical insulation layer 362D covers the conductive traces 366 of the second electrode sensor 339B and the third electrical insulation layer 362C.

In some implementations, it may be beneficial to expose at least a portion of the electrode sensor(s) 139 to an environment that is external to the monopolar electrosurgical electrode 128. In such implementations, one or more of the electrical insulation layers 362A-362B can include an aperture for exposing the portion of the electrode sensor(s) 139. For instance, as shown in FIGS. 3E-3F, the second electrical insulation layer 362B can include a first aperture 368A that exposes a portion of the first electrode sensor 339A to the environment external to the monopolar electrosurgical electrode 128, and the fourth electrical insulation layer 362D can include a second aperture 368B that exposes a portion of the second electrode sensor 339B to the environment external to the monopolar electrosurgical electrode 128.

In FIG. 3E, the first electrode sensor 339A is a temperature sensor. In this example, the first aperture 368A can provide a window through which the first electrode sensor 339A can sense a temperature of the environment and/or an object (e.g., the target tissue). In this arrangement, the portion of the first electrode sensor 339A located within the first aperture 368A can provide a sensing portion of the first electrode sensor 339A for sensing the temperature through the first aperture 368A, and the portion of the first electrode sensor 339A that is covered by the second electrical insulation layer 362B can provide a connector portion of the first electrode sensor 339A for communicating the sensor signal to the electrosurgical tool 112 and/or the electrosurgical generator 110.

Also, in FIG. 3F, the second electrode sensor 339B is an electrochemical sensor. In this example, the second aperture 368B can provide a window through which the second electrode sensor 339B can contact and analyze a substance. In this arrangement, the portion of the second electrode sensor 339B located within the second aperture 368B can provide a sensing portion of the second electrode sensor 339B for sensing a chemical property of a substance through the second aperture 368B, and the portion of the second electrode sensor 339B that is covered by the fourth electrical insulation layer 362D can provide a connector portion of second electrode sensor 339B for communicating the sensor signal to the electrosurgical tool 112 and/or the electrosurgical generator 110.

For instance, FIG. 3F, the second electrode sensor 339B can include a plurality of electrodes for providing the electrochemical sensor such as, for example, (i) a working electrode and (ii) at least one of a counter electrode or a reference electrode. More particularly, in the example shown in FIG. 3F, the conductive traces 366 define a working electrode, a counter electrode, and a reference electrode. However, in another example, the conductive traces 366 can define a different quantity of electrodes (e.g., a working electrode and a counter electrode). Additionally, although not shown in FIG. 3F, the second electrode sensor 339B can include a reagent that is configured to react with a component of a substance and provide for sensing the component of the substance as a result of an electrochemical reaction. As one example, the reagent can include an enzyme that is configured to react with an analyte, which is to be sensed in the substance.

As described above, the second electrical insulation layer 362B and the fourth electrical insulation layer 362D include the first aperture 368A and the second aperture 368B, respectively, in FIGS. 3A-3F. However, in other implementations, an entirety of at least one of the electrode sensor(s) 139 can be embedded in the electrical insulation layers 362A-362D such that no portion of the at least one of the electrode sensor(s) 139 is exposed. For example, the second electrical insulation layer 362B can cover an entirety of the first electrode sensor 339A, and/or the fourth electrical insulation layer 362D can cover an entirety of the second electrode sensor 339B in FIGS. 3E-3F. This may be beneficial for implementations in which it is desirable to inhibit or prevent all of the first electrode sensor 339A and/or all of the second electrode sensor 339B from being exposed to the environment and/or an object external to the monopolar electrosurgical electrode 128. In one example, the second electrical insulation layer 362B can cover the first electrode sensor 339A such that the temperature sensor of the first electrode sensor 339A can sense a temperature of the second electrical insulation layer 362B. In other examples, any of the types of electrode sensor(s) 139 described herein can be partially exposed or completely covered by the electrical insulation layers 362A-362D.

As shown in FIG. 3E, the electrosurgical blade 358B also includes a third electrode sensor 339C on the first side. In this example, the third electrode sensor 339C includes a resistive element 370 having an associated resistance value that provide for determining information relating to the monopolar electrosurgical electrode 128, as described above. In this example, the resistive element 370 is provided in the form of a single conductive trace. The resistance value of the resistance element can be based on a size of the resistance element 370 and a material of the resistance element 370. The conductive trace of the resistive element 370 can be formed from a material that is the same as or different than a material used to form the conductive traces 364, 366 of the first electrode sensor 329A and/or the second electrode sensor 329B. Although the third electrode sensor 339C includes one resistive element 370 in FIG. 3E, the third electrode sensor 339C can include a plurality of resistive elements 370 have a plurality of associated resistance values in other examples, as described above.

In FIG. 3E, the plurality of resistive element 370 is embedded between the first electrical insulation layer 362A and the second electrical insulation layer 362B. More particularly, in FIG. 3E, the second electrical insulation layer 362B entirely covers the resistive element 370 to inhibit or prevent foreign substances from contacting the resistance element 370 and erroneously affecting the resistance value that is sensed by the electrosurgical tool 112 and/or the electrosurgical generator 110 when an electrical stimulus is applied to the resistive element 370.

In one example, the electrosurgical tool 112 and/or the electrosurgical generator 110 can be configured to (i) apply a voltage to the resistance element 370, (ii) responsively sense an electrical parameter (e.g., a current) in a circuit including the resistive element 370, and/or (iii) determine, based on the sensed electrical parameter, the resistance value. The electrosurgical tool 112 and/or the electrosurgical generator 110 can then determine, based on the sensed electrical parameter and/or the determined resistance value, the information relating to the monopolar electrosurgical electrode 128 (e.g., using a look-up table that stores the information in association with possible sensed electrical parameters and/or resistance value).

Referring to FIGS. 3G-3H, an arrangement for communicatively coupling the electrode sensor(s) 139 of the monopolar electrosurgical electrode 128 with a receptacle 372 of the electrosurgical tool 112 is shown according to an example. As noted above, the electrosurgical tool 112 can include the receptacle 372 that can receive and electrically couple the shank portion 358A of the monopolar electrosurgical electrode 128 to the electrosurgical tool 112. As shown in FIG. 3G, the receptacle 372 can include one or more receptacle contacts 374 that are configured to electrically couple with one or more sensor contacts 376, which are shown in FIG. 3H with the shank portion 358A omitted for clarity of illustration. In this arrangement, the electrode sensor(s) 139 can transmit the sensor signal to the electrosurgical tool 112 and/or the electrosurgical generator 110 via the electrical coupling between the receptacle contact(s) 374 and the sensor contact(s) 376.

In one implementation, the receptacle 372 and the shank portion 358A can be configured such that the receptacle contact(s) 374 engage the sensor contact(s) 376 due to a friction-fit between the receptacle 372 and the shank portion 358A. In another example, the receptacle 372 and/or the shank portion 358A can include one or more springs for biasing the receptacle contact(s) 374 towards the sensor contact(s) 376 and/or biasing the sensor contact(s) 376 towards the receptacle contact(s) 374.

Although the receptacle 372 includes four receptacle contacts 374 and the shank portion 358A includes four sensor contacts 376 in FIGS. 3G-3H, the receptacle 372 and the shank portion 358A can include a different quantity of receptacle contacts 374 and/or a different quantity of sensor contacts 376 in other examples.

Although the monopolar electrosurgical electrode 128 shown in FIGS. 3A-3F includes three electrode sensors 139, the monopolar electrosurgical electrode 128 can include a different quantity of electrode sensors 139 in other examples (e.g., a single electrode sensor 139, two electrode sensors 139, four electrode sensors 139, five electrode sensors 139, etc.). Additionally, although the first electrode sensor 339A is a temperature sensor and the second electrode sensor 339B is an electrochemical sensor in the implementation shown in FIGS. 3A-3F, the monopolar electrosurgical electrode 128 can include different types of the electrode sensors 139 and/or combinations of the electrode sensors 139 described above with respect to FIG. 1 in other examples. Further, although the monopolar electrosurgical electrode 128 included the resistive elements 370 in the implementation shown in FIGS. 3A-3F, the monopolar electrosurgical electrode 128 can omit the resistive elements 370 in other examples.

Additionally, as described above, one or more of the electrical insulation layers 364A-364D can include an aperture (e.g., the first aperture 368A or the second aperture 368B) to expose a portion of the electrode sensor(s) 139. In the examples shown in FIGS. 3A-3F, the first electrode sensor 339A and the second electrode sensor 339B each included a single sensing portion that was exposed by a single aperture 368A, 368B. However, in other examples, a single electrode sensor 139 can include a plurality of sensing portions that are each exposed at a respective aperture of a plurality of apertures in the electrical insulation layers.

As one example, FIG. 4 depicts another implementation of the monopolar electrosurgical electrode 128 in which the electrode sensor 139 is a depth sensor that is configured to sense a depth that the electrosurgical blade 358B is inserted into tissue. In FIG. 4, the electrode sensor 139 is embedded between the electrical insulation layers 462 (e.g., between the first electrical insulation layer 362A and the second electrical insulation layer 362B shown and described above with respect to FIGS. 3D-3E). As shown in FIG. 4, the electrode sensor 139 can include a plurality of conductive elements 464A, 464B (e.g., conductive traces) that are not electrically coupled to each other by any other component of the monopolar electrosurgical electrode 128. The conductive elements 464A, 464B can be embedded between the electrical insulation layers 464.

As shown in FIG. 4, the electrical insulation layers 462 can include a plurality of apertures 468A-468C at a plurality of positions along the length of the monopolar electrosurgical electrode 128 (e.g., the dimension extending between the proximal end 328A and the distal end 328B in FIG. 4). The apertures 468A-468C can each expose a respective portion of the conductive elements 464A, 464B along the length. In this arrangement, when a substance (e.g., tissue) contacts the conductive elements 464A, 464B at one or more of the apertures 468A-468C, the substance can electrically couple the conductive elements 464A, 464B to each other. When the electrosurgical tool 112 and/or the electrosurgical generator 110 applies a voltage to the conductive elements 464A, 464B, an electrical circuit is formed by the conductive elements 464A, 464B and the substance coupling the conductive elements 464A, 464B. The resistance of this electrical circuit is based on a location at which the substance couples the conductive elements 464A, 464B. When the distal end 328B of the monopolar electrosurgical electrode 128 is inserted in tissue, a depth at which the electrosurgical blade 358B is inserted in the tissue can be determined by the electrosurgical tool 112 and/or the electrosurgical generator 110 in manner similar to that described above for the resistive element 370 (e.g., by applying a voltage, sensing the electrical parameter, determining the resistance value based on the electrical parameter, and determining the depth based on the determined resistance value).

Although the sensing portions of the electrode sensor(s) 139 are located at the apertures 368A, 368B, 468A, 468B, 468C in the examples described above, the sensing portions of the electrode sensor(s) 139 can be covered by the electrical insulation layers 362A-362D, 462 in other examples. More generally, each electrode sensor 139 can include at least one sensing portion that is configured to sense the one or more operational conditions during an electrosurgical procedure, and at least one connector portion that is configured to transmit the sensor signals that can provide a basis for feedback control of an electrosurgical system to improve the electrosurgical procedure. The sensing portion(s) can be located distal of the connector portion(s) such that the sensor signal, which is indicative of the one or more conditions sensed by the sensing portion, is transmitted in a proximal direction along the electrosurgical blade 358B.

Referring now to FIG. 5, a perspective view of another implementation of the electrosurgical tool 112 is shown according to an example. In particular, FIG. 5 shows an implementation of the electrosurgical tool 112 that (i) includes illumination features and (ii) in which the shaft 126 is axially moveable and rotatable relative to the handle 124. As shown in FIG. 5, the electrosurgical tool 112 includes the housing 123 defining the interior bore 125, the shaft 126 telescopically moveable in the interior bore 125 of the housing 123, and the monopolar electrosurgical electrode 128 coupled to the shaft 126.

Additionally, in FIG. 5, the optical structure 142 is at a distal end 578 of the shaft 126. In this arrangement, the optical structure 142 can telescopically move with the shaft 126 relative to the housing 123. In FIG. 5, the optical structure 142 extends around the monopolar electrosurgical electrode 128. This can help to emit the light in a relatively uniform manner by reducing (or preventing) shadows due to an orientation of the optical structure 142 and the monopolar electrosurgical electrode 128 relative to the surgical site. However, in other examples, the optical structure 142 may not extend entirely around the monopolar electrosurgical electrode 128 at the distal end 578 of the shaft 126, and/or the optical structure 142 can be at a different position on the shaft 126 and/or the housing 123.

In some examples, the electrosurgical tool 112 can include a collar 580 at a proximal end of the housing 123. The collar 580 can be rotatable relative to the housing 123 to increase and/or decrease friction between an outer surface of the shaft 126 and an inner surface of the collar 580. In this way, the collar 580 to allow and/or inhibit axial telescopic movement of the shaft 126 relative to the housing 123.

As shown in FIG. 5, the electrosurgical tool 112 includes the power cord 122. At a proximal end 584 of the power cord 122, the power cord 122 includes a plug 586 configured to couple to the connector 120 of the electrosurgical generator 110. A distal end of the power cord 122 is coupled to the printed circuit board 132 in an interior cavity of the housing 123. In this arrangement, the power cord 122 extends proximally from the housing 123 to the plug 586.

Additionally, as shown in FIG. 5, the user input device(s) 130 include a first button 530A, a second button 530B, and a third button 530C on an exterior surface of the housing 123. In one implementation, the first button 530A can be actuated to operate the electrosurgical tool 112 in a cutting mode of operation, the second button 530B can be actuated to operate the electrosurgical tool 112 in a coagulation mode of operation, and the third button 530C can be actuated to operate the light source 140 (i.e., to cause the light source 140 to emit light or cease emitting light). As described above, the user input device(s) 130 can be configured differently in other examples. For instance, the electrosurgical tool 112 can be operable in a lesser quantity of modes of operation, a greater quantity of modes of operation, and/or different types of modes of operation in other examples (e.g., such as the example modes of operation described above). Additionally, for instance, the at least one user input device 130 can additionally or alternatively include the user interface 116 of the electrosurgical generator 110 and/or another external device (e.g., a footswitch) for operating the electrosurgical tool 112 in one or more modes of operation.

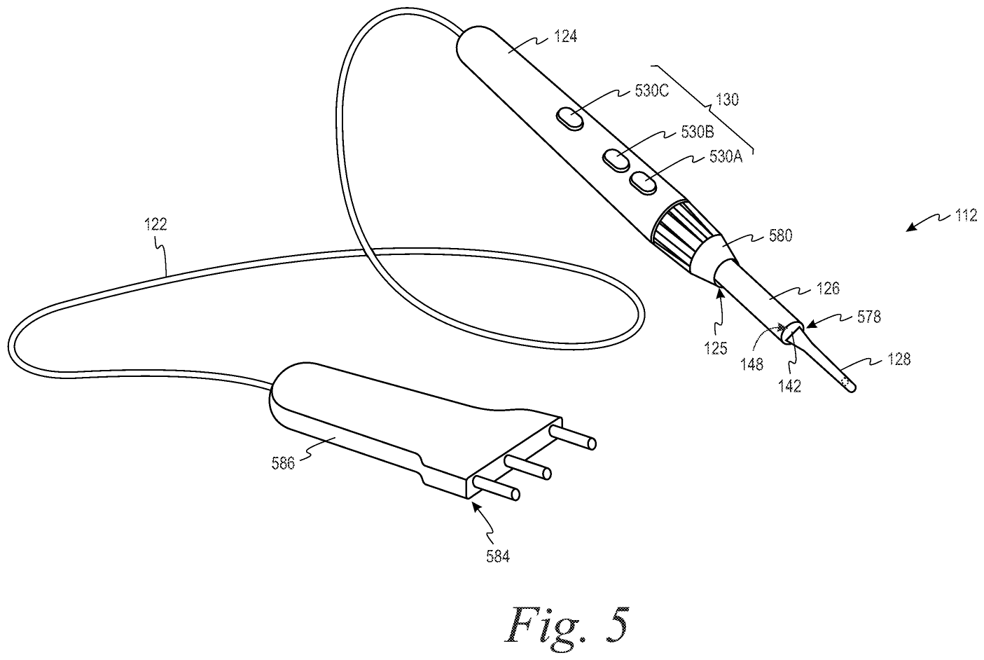

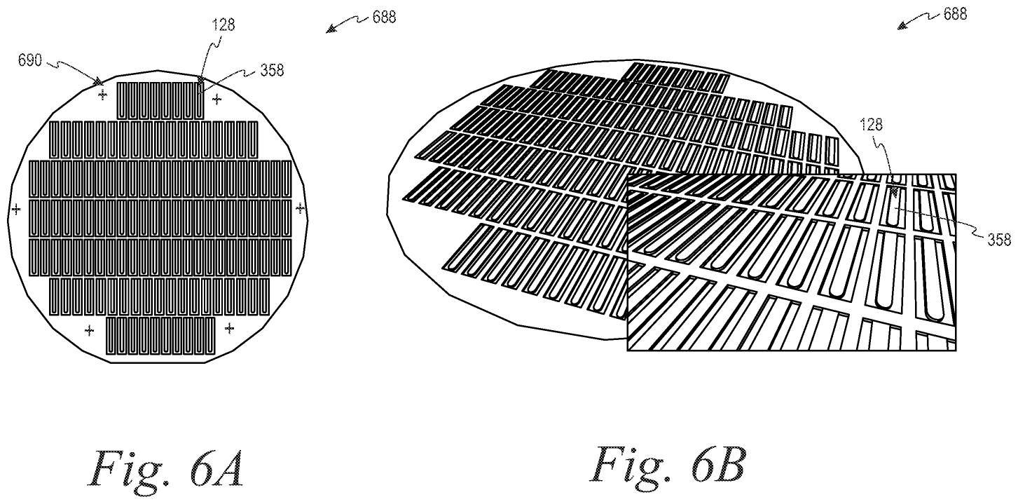

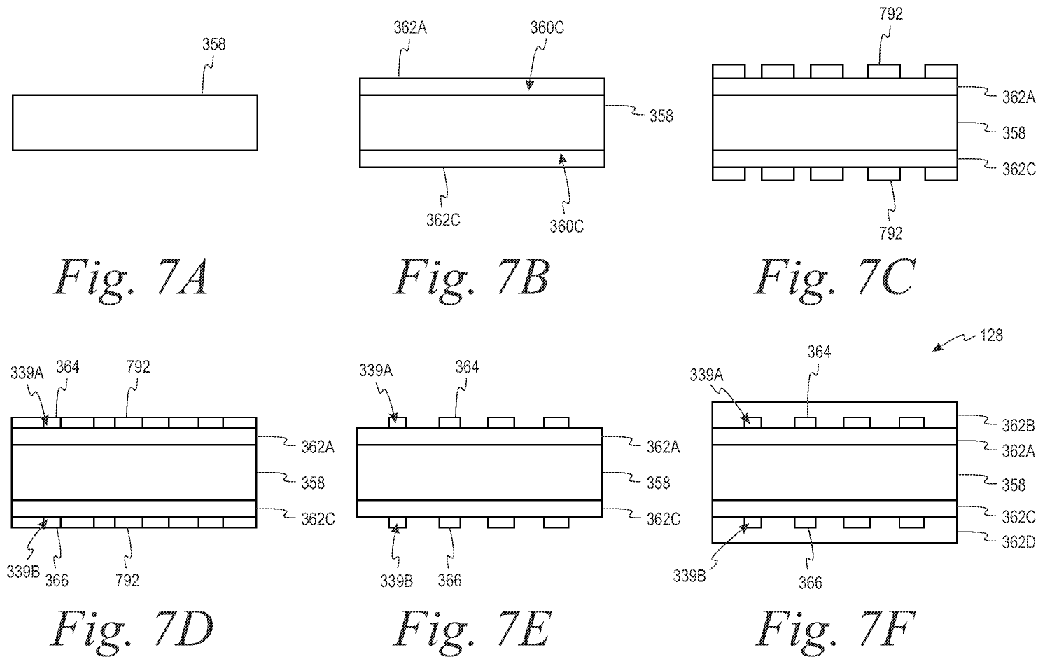

Referring now to FIGS. 6A-6B and 7A-7F, a process for forming the monopolar electrosurgical electrode 128 is shown according to an example. In particular, FIG. 6A-6B depicts a wafer 688 for batch fabrication of a plurality of the monopolar electrosurgical electrodes 128, and FIGS. 7A-7F depict cross-sectional views of one electrosurgical blade 358B within the wafer 688 at different stages of the fabrication process, according to an example.

The wafer 688 provides the electrosurgical substrate 358 for each monopolar electrosurgical electrode 128 of the plurality of monopolar electrosurgical electrodes 128 formed from the wafer 688. Accordingly, the wafer 688 can be a stainless steel wafer in an example in which the electrosurgical substrate 358 is formed from stainless steel. The wafer 688 can be formed from other materials in examples in which the electrosurgical substrate 358 is formed from a material other than stainless steel.

In one example, the wafer 688 can have a diameter of approximately 200 millimeters (mm) and a thickness of approximately 500 microns. This can help to repurpose semiconductor foundry tools for new uses in a manufacturing process for the monopolar electrosurgical electrode 128 described herein.

In the example shown in FIGS. 7A-7F, the fabrication process can first include forming, providing, and/or receiving the wafer 688 shown in FIGS. 6A-6B. FIG. 7A shows the electrosurgical substrate 358 for one of the monopolar electrosurgical electrodes 128 on the wafer 688 shown in FIGS. 6A-6B. Next, as shown in FIG. 7B, the fabrication process can include forming the first electrical insulation layer 362A on the first major surface 360C, and forming the third electrical insulation layer 362C on the second major surface 360D. In one implementation, forming the first electrical insulation layer 362A can include forming a first passivation layer and forming the third electrical insulation layer 362C can include forming a third passivation layer. However, in other examples, forming the first electrical insulation layer 362A and the third electrical insulation layer 362C can include at least one of performing at least one process selected from a group consisting of: (i) a plasma vapor deposition process, (ii) a chemical vapor deposition process, (iii) a printing process, (iv) a spin coating process, (v) a spray coating process, (vi) a sintering process, and (vii) a thermal oxidation process.

After forming the first electrical insulation layer 362A and the third electrical insulation layer 362C, the fabrication process can include forming the electrode sensor(s) 139 using a lithography process. In one example, the process of forming the wafer 688 can include forming one or more alignment marks 690 on the wafer 688 such that the lithography process can be performed based on the one or more alignment marks 690.

FIGS. 7C-7E show stages of the lithography process for forming the first electrode sensor 339A and the second electrode sensor 339B of FIG. 3D, according to an example. For instance, FIG. 7C shows a lithographic photomask 792 applied to the first electrical insulation layer 362A and the third electrical insulation layer 362C. As shown in FIG. 7C, the lithographic photomask 792 defines an exposed portion on the first electrical insulation layer 362A and the third electrical insulation layer 362C at which the conductive traces 364, 366 for the first electrode sensor 339A and the second electrode sensor 339B can be formed. FIG. 7D shows the conductive traces 364, 366 formed in the exposed portions defined by the lithographic photomask 792. FIG. 7E shows the conductive traces 364, 366 of the first electrode sensor 339A and the second electrode sensor 339B after removing the lithographic photomask.

After forming the electrode sensor(s) 139 using lithography, the fabrication process can include forming the second electrical insulation layer 362B on the first electrode sensor 339A and the first electrical insulation layer 362A such that the first electrode sensor 339A is embedded between the first electrical insulation layer 362A and the second electrical insulation layer 362B as shown in FIG. 7F. Similarly, in FIG. 7F, the fabrication process can include forming the fourth electrical insulation layer 362D on the second electrode sensor 339B and the third electrical insulation layer 362C such that the second electrode sensor 339B is embedded between the third electrical insulation layer 362C and the fourth electrical insulation layer 362D.

After forming the electrode sensor(s) 139 and the electrical insulation layers 362A-362D, the monopolar electrosurgical electrode 128 can be removed from the wafer 688. For instance, in one example, each monopolar electrosurgical electrode 128 can be cut from the wafer 688 using a laser.

In another example, instead of forming the electrode sensor(s) 139 on the wafer 688, a fabrication process can include (i) forming the electrode sensor(s) 139 on a silicon carrier wafer, (ii) transferring the electrode sensor(s) from the silicon carrier wafer to the electrosurgical substrate, and (iii) after transferring the electrode sensor(s) to the electrosurgical substrate 358 on the wafer 688, cutting the electrosurgical substrate 358 out of the wafer 688. The fabrication process used may be based on the type of electrode sensor 139 that is be incorporated into the monopolar electrosurgical electrode 128 as different types of electrode sensors 139 can be more easily and efficiently formed by different fabrication processes.

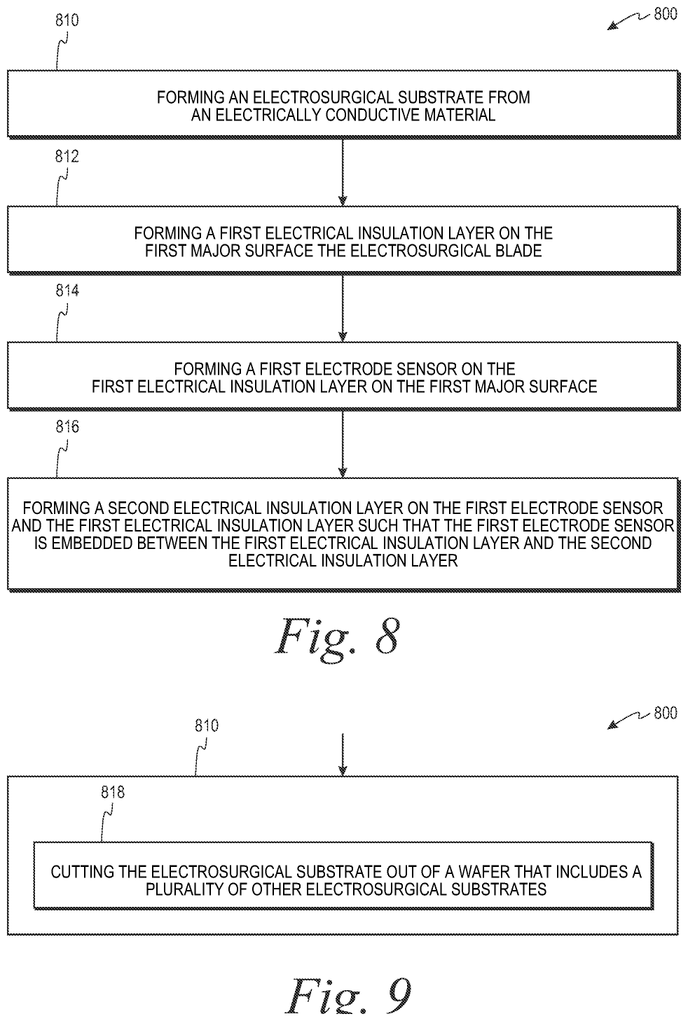

Referring now to FIG. 8, a flowchart of a process 800 for forming a monopolar electrosurgical electrode is shown according to an example. As shown in FIG. 8, at block 810, the process 800 can include forming an electrosurgical substrate from an electrically conductive material. The electrosurgical substrate can include an electrosurgical blade. The electrosurgical blade can include a first lateral surface, a second lateral surface opposite the first lateral surface, a first major surface extending between the first lateral surface and the second lateral surface on a first side of the electrosurgical blade, and a second major surface extending between the first lateral surface and the second lateral surface on a second side of the electrosurgical blade that is opposite the first side.

At block 812, the process 800 can also include forming a first electrical insulation layer on the first major surface the electrosurgical blade. At block 814, the process 800 can further include forming a first electrode sensor on the first electrical insulation layer on the first major surface. At block 816, the process 800 can include forming a second electrical insulation layer on the first electrode sensor and the first electrical insulation layer such that the first electrode sensor is embedded between the first electrical insulation layer and the second electrical insulation layer.

FIGS. 9-17 depict additional aspects of the process 800 according to further examples. As shown in FIG. 9, forming the electrosurgical substrate at block 810 can include cutting the electrosurgical substrate out of a wafer that includes a plurality of other electrosurgical substrates at block 818. In some examples, the wafer can include a stainless steel wafer, the wafer can have a thickness of approximately 500 microns, and/or the wafer can have a diameter of approximately 200 millimeters (mm).

As shown in FIG. 10, forming the first electrical insulation layer at block 812 can include at least one of performing at least one process selected from a group consisting of: (i) a plasma vapor deposition process, (ii) a chemical vapor deposition process, (iii) a printing process, (iv) a spin coating process, (v) a spray coating process, (vi) a sintering process, and (vii) a thermal oxidation process at block 820.

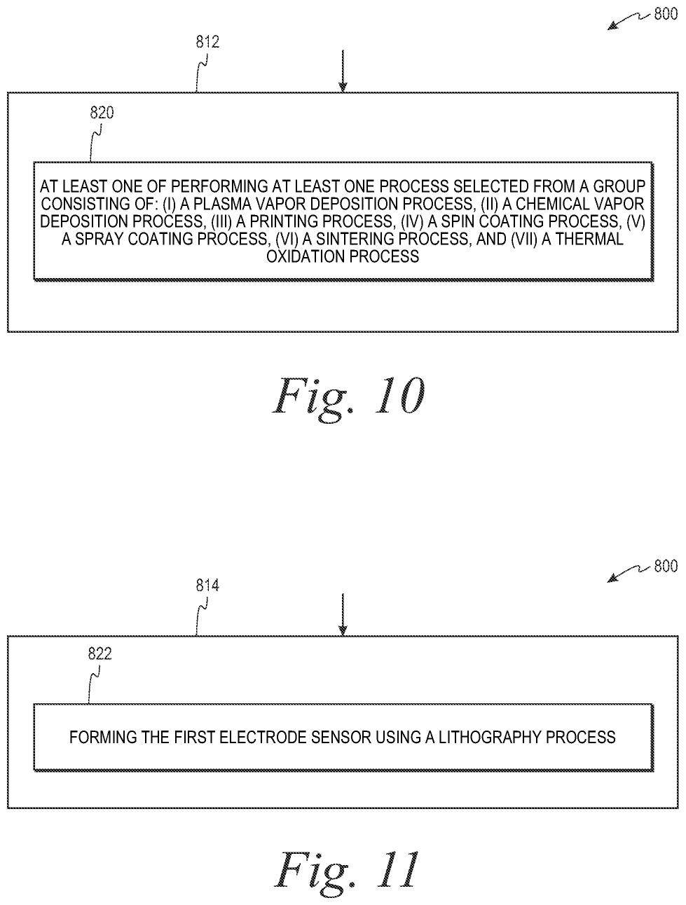

As shown in FIG. 11, forming the first electrode sensor at block 814 can include forming the first electrode sensor using a lithography process at block 822.

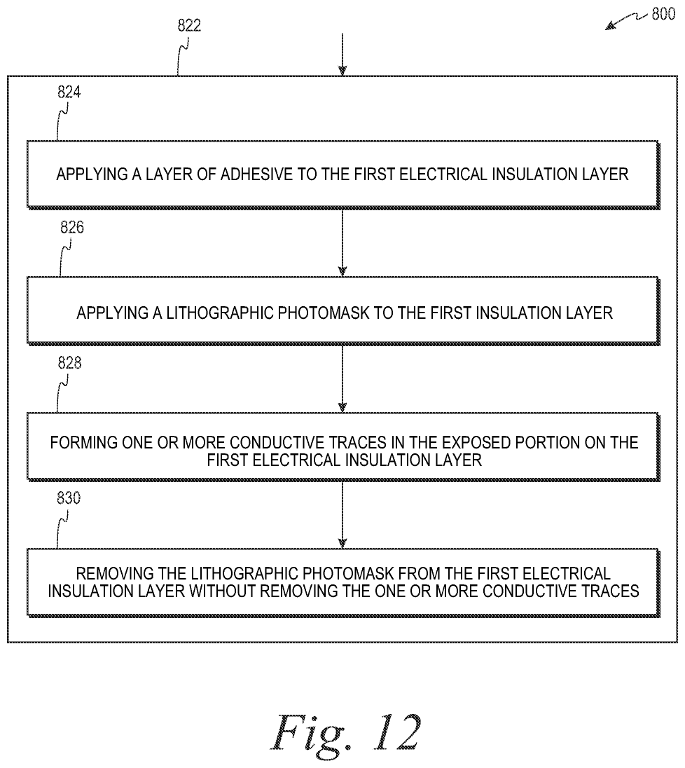

As shown in FIG. 12, forming the first electrode sensor using a lithography process at block 822 can include applying a layer of adhesive to the first electrical insulation layer at block 824. The lithography process can also include applying a lithographic photomask to the first insulation layer at block 826. The lithographic photomask defines an exposed portion on the first electrical insulation layer. After applying the lithographic photomask at block 826, the lithography process can include forming one or more conductive traces in the exposed portion on the first electrical insulation layer at block 828. After forming the one or more conductive traces at block 828, the lithography process can include removing the lithographic photomask from the first electrical insulation layer without removing the one or more conductive traces at block 830.

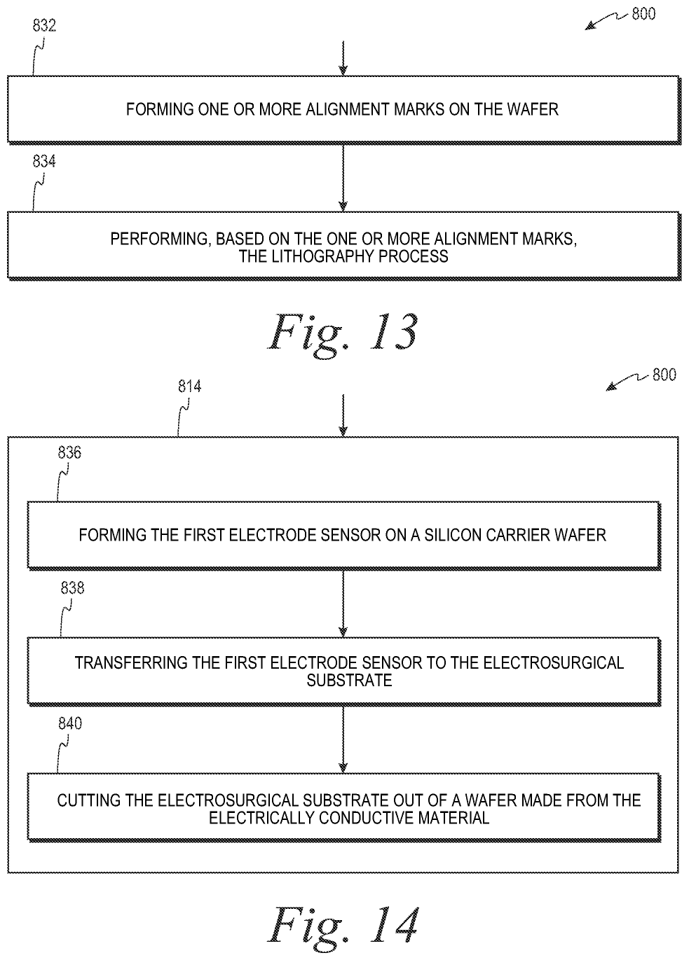

As shown in FIG. 13, the process 800 can also include forming one or more alignment marks on the wafer at block 832, and performing, based on the one or more alignment marks, the lithography process at block 834.

As shown in FIG. 14, forming the first electrode sensor at block 814 can include forming the first electrode sensor on a silicon carrier wafer at block 836. After forming the first electrode sensor on the silicon carrier wafer, forming the first electrode sensor can include transferring the first electrode sensor to the electrosurgical substrate at block 838. After transferring the first electrode sensor to the electrosurgical substrate, forming the first electrode sensor can include cutting the electrosurgical substrate out of a wafer made from the electrically conductive material at block 840.

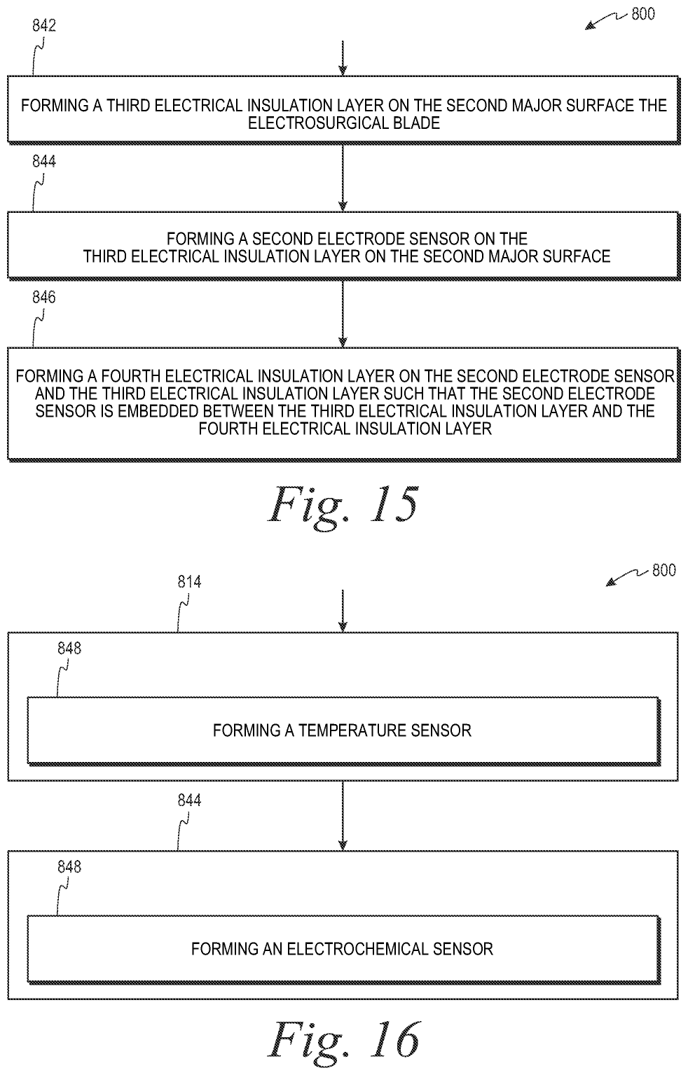

As shown in FIG. 15, the process 800 can also include forming a third electrical insulation layer on the second major surface the electrosurgical blade at block 842, forming a second electrode sensor on the third electrical insulation layer on the second major surface at block 844, and forming a fourth electrical insulation layer on the second electrode sensor and the third electrical insulation layer such that the second electrode sensor is embedded between the third electrical insulation layer and the fourth electrical insulation layer at block 846.

As shown in FIG. 16, forming the first electrode sensor at block 814 can include forming a temperature sensor at block 848. Also, as shown in FIG. 16, forming the second electrode sensor at block 844 can include forming an electrochemical sensor at block 850.

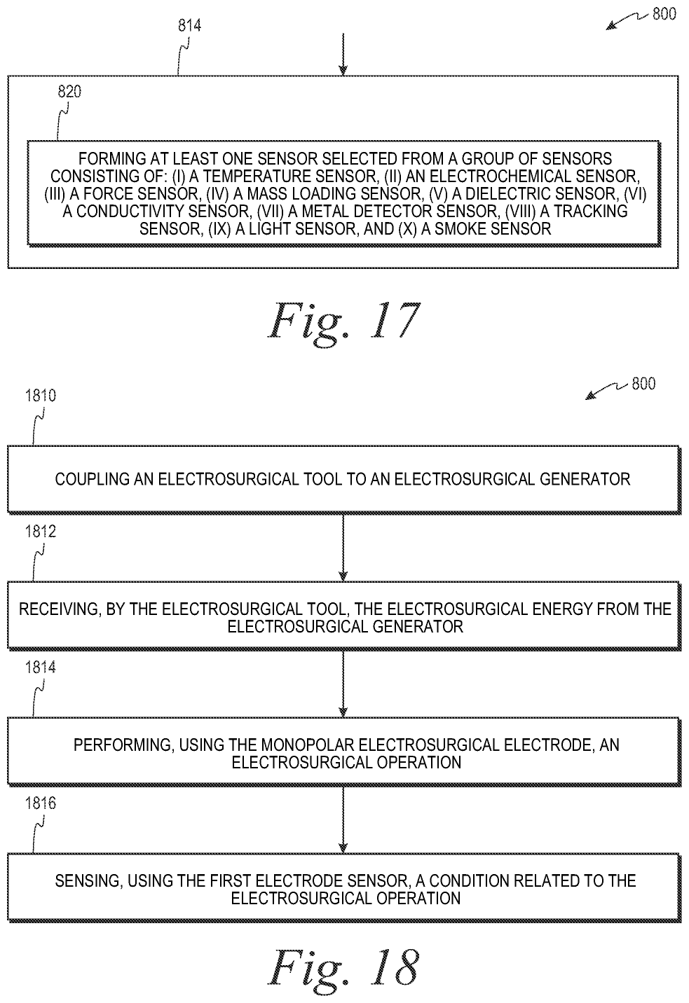

As shown in FIG. 17, forming the first electrode sensor at block 814 can include forming at least one sensor selected from a group of sensors consisting of: (i) a temperature sensor, (ii) an electrochemical sensor, (iii) a force sensor, (iv) a mass loading sensor, (v) a dielectric sensor, (vi) a conductivity sensor, (vii) a metal detector sensor, (viii) a tracking sensor, (ix) a light sensor, and (x) a smoke sensor at block 852.

Referring now to FIG. 18, a flowchart of a process 1800 for performing electrosurgery is shown according to an example. As shown in FIG. 18, at block 1810, the process 1800 includes coupling an electrosurgical tool to an electrosurgical generator. The electrosurgical tool includes a monopolar electrosurgical electrode. The monopolar electrosurgical electrode includes an electrosurgical substrate including an electrically conductive material extending in an axial direction from a proximal end to a distal end. The proximal end is configured to receive electrosurgical energy from an electrosurgical tool. The electrosurgical substrate includes an electrosurgical blade that is configured for at least one of cutting or coagulation of tissue by the electrosurgical energy received from the electrosurgical tool.

The electrosurgical blade includes: (i) a first lateral surface, (ii) a second lateral surface opposite the first lateral surface, (iii) a first major surface extending between the first lateral surface and the second lateral surface on a first side of the electrosurgical blade, (iv) a second major surface extending between the first lateral surface and the second lateral surface on a second side of the electrosurgical blade that is opposite the first side, and (v) a first electrode sensor embedded between a plurality of electrical insulation layers on the first major surface of the electrosurgical blade.

At block 1812, the process 1800 can include receiving, by the electrosurgical tool, the electrosurgical energy from the electrosurgical generator. Responsive to receiving the electrosurgical energy at block 1812, the process 1800 can include performing, using the monopolar electrosurgical electrode, an electrosurgical operation at block 1814. At block 1816, the process 1800 can include sensing, using the first electrode sensor, a condition related to the electrosurgical operation.

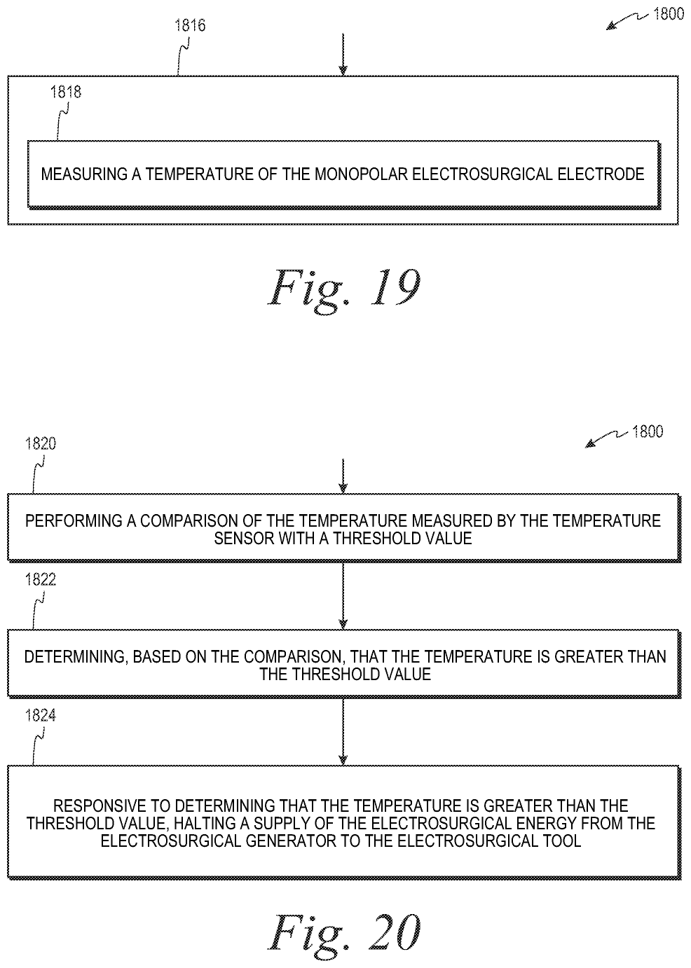

FIGS. 19-35 depict additional aspects of the process 1800 according to further examples. In FIG. 19, the first electrode sensor can be a temperature sensor, and sensing the condition at block 1816 can include measuring a temperature of the monopolar electrosurgical electrode at block 1818.

As shown in FIG. 20, the process 1800 can also include performing a comparison of the temperature measured by the temperature sensor with a threshold value at block 1820. The process 1800 can further include determining, based on the comparison, that the temperature is greater than the threshold value at block 1822. Responsive to determining that the temperature is greater than the threshold value at block 1822, the process 1800 can include halting a supply of the electrosurgical energy from the electrosurgical generator to the electrosurgical tool at block 1824. In one example, the threshold value can be less than or equal to a melting temperature of at least one of the plurality of electrical insulation layers.

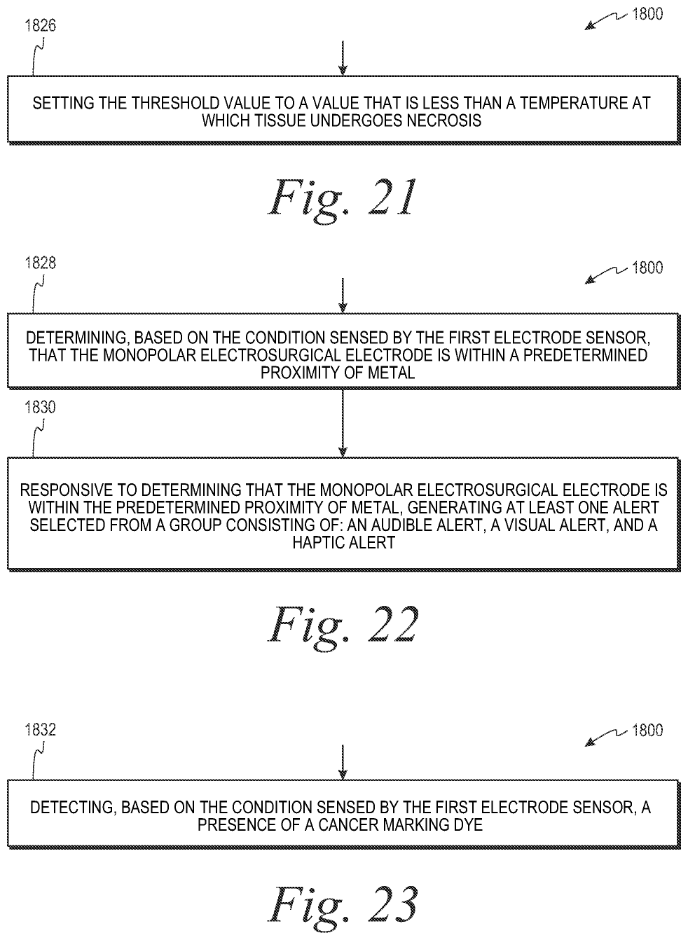

As shown in FIG. 21, the process 1800 can also include setting the threshold value to a value that is less than a temperature at which tissue undergoes necrosis at block 1826.

As shown in FIG. 22, the process 1800 can further include determining, based on the condition sensed by the first electrode sensor, that the monopolar electrosurgical electrode is within a predetermined proximity of metal at block 1828. Responsive to determining that the monopolar electrosurgical electrode is within the predetermined proximity of metal at block 1828, the process 1800 can include generating at least one alert selected from a group consisting of: an audible alert, a visual alert, and a haptic alert at block 1830.

As shown in FIG. 23, the process 1800 can also include detecting, based on the condition sensed by the first electrode sensor, a presence of a cancer marking dye at block 1832. In an example of the process 1800 shown in FIG. 23, the first electrode sensor can be an electrochemical sensor.

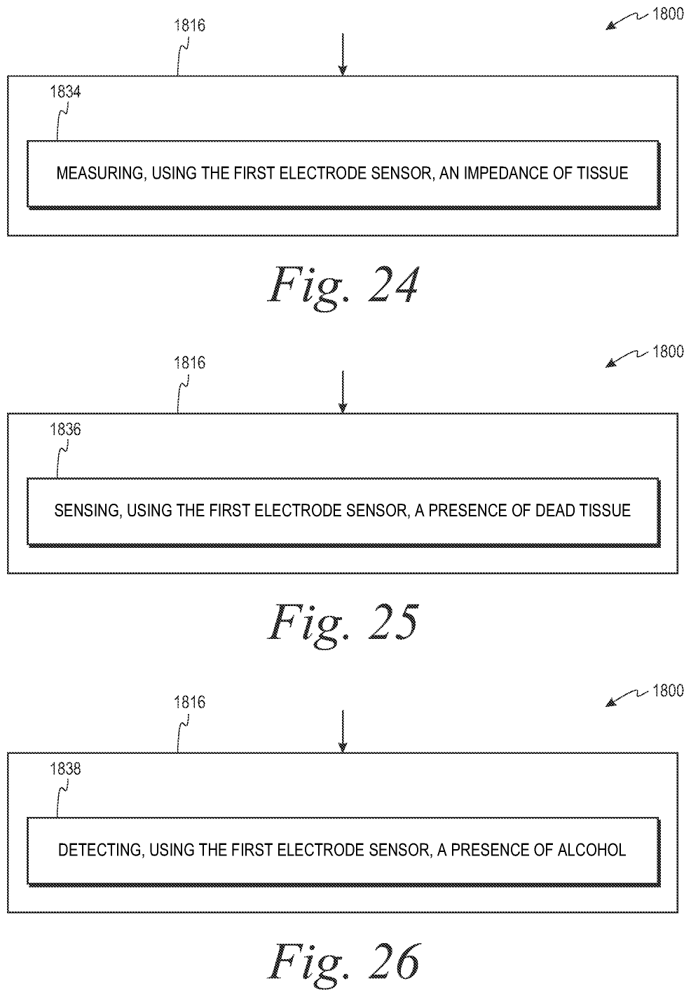

As shown in FIG. 24, sensing the condition at block 1816 can include measuring, using the first electrode sensor, an impedance of tissue at block 1834. In an example of the process 1800 shown in FIG. 24, the first electrode sensor can be an electrochemical sensor. In some examples, measuring the impedance of the tissue at block 1834 can be performed while simultaneously cutting the tissue using the electrosurgical energy.

As shown in FIG. 25, sensing the condition at block 1816 can include sensing, using the first electrode sensor, a presence of dead tissue at block 1836. In an example of the process 1800 shown in FIG. 25, the first electrode sensor can be an electrochemical sensor.

As shown in FIG. 26, sensing the condition at block 1816 can include detecting, using the first electrode sensor, a presence of alcohol at block 1838. In an example of the process 1800 shown in FIG. 26, the first electrode sensor can be an electrochemical sensor.

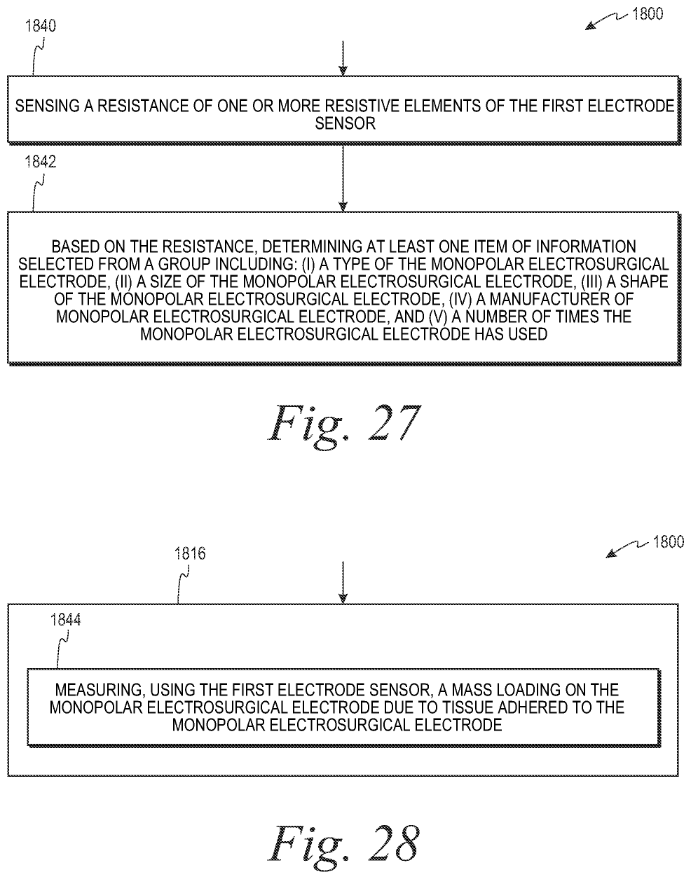

As shown in FIG. 27, the process 1800 can also include sensing a resistance of one or more resistive elements of the first electrode sensor at block 1840 and, based on the resistance, determining at least one item of information selected from a group including: (i) a type of the monopolar electrosurgical electrode, (ii) a size of the monopolar electrosurgical electrode, (iii) a shape of the monopolar electrosurgical electrode, (iv) a manufacturer of monopolar electrosurgical electrode, and (v) a number of times the monopolar electrosurgical electrode has used at block 1842.

As shown in FIG. 28, sensing the condition at block 1816 can include measuring, using the first electrode sensor, a mass loading on the monopolar electrosurgical electrode due to tissue adhered to the monopolar electrosurgical electrode at block 1844.

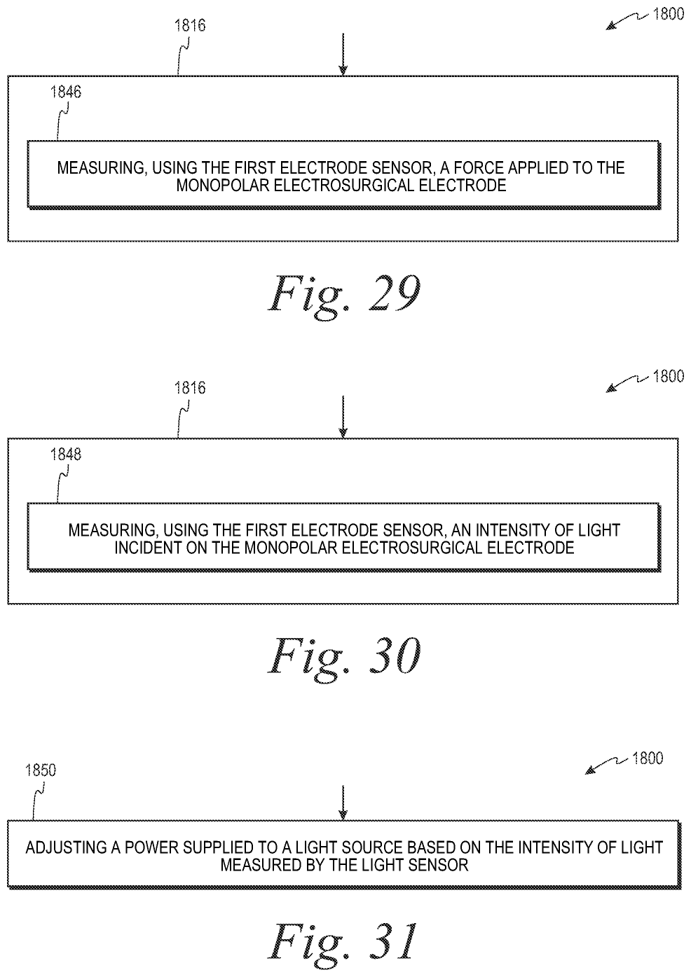

As shown in FIG. 29, sensing the condition at block 1816 can include measuring, using the first electrode sensor, a force applied to the monopolar electrosurgical electrode at block 1846.

As shown in FIG. 30, sensing the condition at block 1816 can include measuring, using the first electrode sensor, an intensity of light incident on the monopolar electrosurgical electrode at block 1848. In an example of the process 1800 shown in FIG. 30, the first electrode sensor can be a light sensor.

As shown in FIG. 31, the process 1800 can also include adjusting a power supplied to a light source based on the intensity of light measured by the light sensor at block 1850.

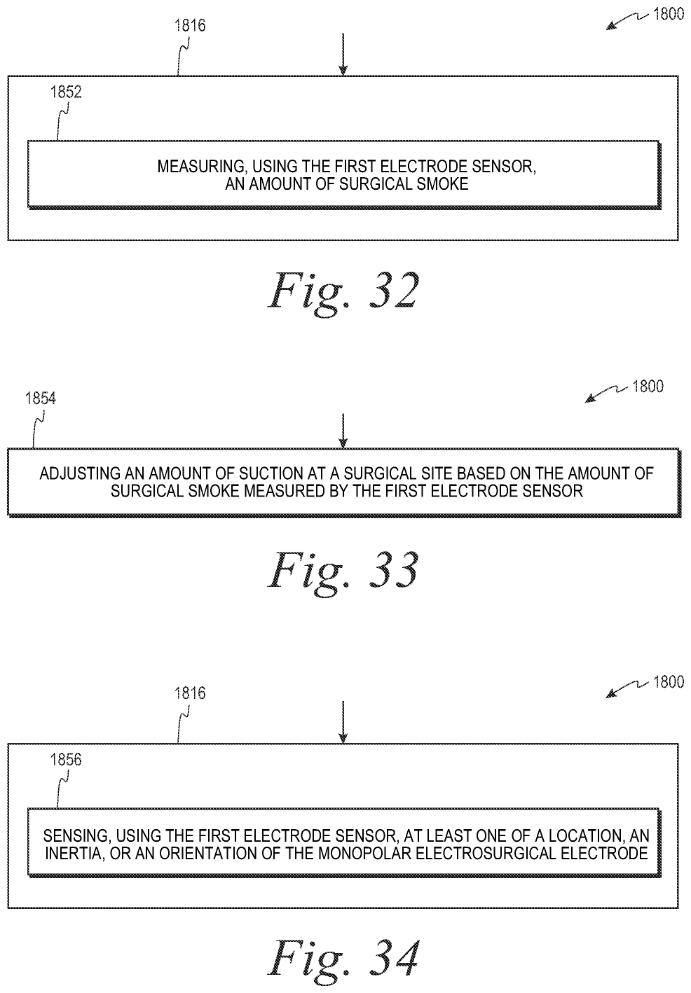

As shown in FIG. 32, sensing the condition at block 1816 can include measuring, using the first electrode sensor, an amount of surgical smoke at block 1852.

As shown in FIG. 33, the process 1800 can also include adjusting an amount of suction at a surgical site based on the amount of surgical smoke measured by the first electrode sensor at block 1854.

As shown in FIG. 34, sensing the condition at block 1816 can include sensing, using the first electrode sensor, at least one of a location, an inertia, or an orientation of the monopolar electrosurgical electrode at block 1856.

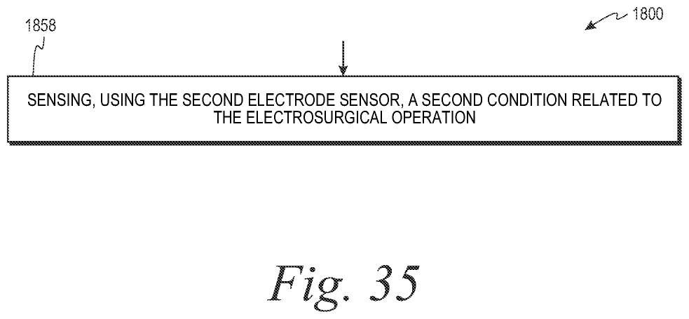

In FIG. 35, the monopolar electrosurgical electrode can further include a second electrode sensor embedded between the plurality of electrical insulation layers on the second major surface of the electrosurgical blade. As shown in FIG. 35, the process 1800 can also include sensing, using the second electrode sensor, a second condition related to the electrosurgical operation at block 1858. In one example, the first electrode sensor can include a temperature sensor and the second electrode sensor can include an electrochemical sensor.

It should be understood that for these and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, one or more blocks or portions of the block(s) in FIGS. 8-35, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

Additionally, in some examples described above, the electrode sensor(s) 139 transmit the sensor signal(s) to the electrosurgical generator 110 for processing (e.g., by the controller 141) and/or user interface 116 can generate an alert based, at least in part, on the sensor signal. However, in other examples, the electrosurgical tool 112 can include a controller that can additionally or alternatively receive and process the sensor signal(s) as described above with respect to the electrosurgical generator 110 and/or the controller 141. Also, the electrosurgical tool 112 can additionally or alternatively include an output device that can generate, based at least in part on the sensor signal, audible alert, a visual alert, and a haptic alert as described above with respect to the user interface 116 of the electrosurgical generator 110.

Additionally, in the examples described above, the monopolar electrosurgical electrode 128 is a blade-type electrode. However, in other examples, the concepts and features described herein can be implemented in other types of monopolar electrosurgical electrodes such as, for example, a ball-shaped electrode, a loop-shaped electrode, and/or a cone-shaped electrode.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present application is not to be limited by the subject specification, but rather only by the plain meaning of the claim terms employed.

What is claimed is:

1. A monopolar electrosurgical electrode, comprising:
an electrosurgical substrate comprising an electrically conductive material extending in an axial direction from a proximal end to a distal end, wherein the proximal end is configured to receive electrosurgical energy from an electrosurgical tool, wherein the electrosurgical substrate comprises an electrosurgical blade that is configured for at least one of cutting or coagulation of tissue by the electrosurgical energy received from the electrosurgical tool, wherein the electrosurgical blade comprises:
a first lateral surface,
a second lateral surface opposite the first lateral surface,
a first major surface extending between the first lateral surface and the second lateral surface on a first side of the electrosurgical blade;
a second major surface extending between the first lateral surface and the second lateral surface on a second side of the electrosurgical blade that is opposite the first side; and
a first electrode sensor embedded between a plurality of electrical insulation layers on the first major surface of the electrosurgical blade.

2. The monopolar electrosurgical electrode of claim 1, wherein the first electrode sensor is a sensor selected from a group of sensors consisting of: (i) a temperature sensor, (ii) an electrochemical sensor, (iii) a force sensor, (iv) a mass loading sensor, (v) a dielectric sensor, (vi) a conductivity sensor, (vii) a metal detector sensor, (viii) a tracking sensor configured to sense at least one of: a location of the monopolar electrosurgical electrode and an orientation of the monopolar electrosurgical electrode, (ix) light sensor, and (x) a smoke detector sensor.

3. The monopolar electrosurgical electrode of any one of claims 1-2, further comprising a second electrode sensor embedded between the plurality of electrical insulation layers on the second major surface of the electrosurgical blade.

4. The monopolar electrosurgical electrode of claim 3, wherein the first electrode sensor comprises a temperature sensor and the second electrode sensor comprises an electrochemical sensor.

5. The monopolar electrosurgical electrode of any one of claims 1-4, wherein the plurality of electrical insulation layers comprise:
a first electrical insulation layer covering the first major surface of the electrosurgical blade, wherein the first electrode sensor comprises a plurality of conductive traces on the first electrical insulation layer; and
a second electrical insulation layer covering the plurality of conductive traces of the first electrode sensor and the first electrical insulation layer.

6. The monopolar electrosurgical electrode of any one of claims 1-5, wherein the first electrode sensor comprises an electrochemical sensor,
wherein the electrochemical sensor comprises a plurality of electrodes, and
wherein the plurality of electrodes comprise (i) a working electrode and (ii) at least one of a counter electrode or a reference electrode.

7. The monopolar electrosurgical electrode of claim 6, wherein the second electrical insulation layer defines an aperture at the plurality of electrodes such that the plurality of electrodes are exposed.

8. The monopolar electrosurgical electrode of any one of claims 1-7, wherein the first electrode sensor comprises at least one sensing portion and at least one connector portion,
wherein the at least one sensing portion is configured to sense one or more operational conditions during an electrosurgical procedure, and
wherein the at least one connector portion is configured to transmit a sensor signal, which is indicative of the one or more conditions sensed by the sensing portion, in a proximal direction along the electrosurgical blade.

9. The monopolar electrosurgical electrode of any one of claims 1-8, wherein the plurality of electrical insulation layers are formed from one or more materials selected from a group of materials consisting of: aluminum oxide (Al₂O₃), silicon dioxide (SiO₂), silicon nitride (Si₃N₄), silicon oxynitride (SiON), and tantalum pentoxide (Ta₂O₅).

10. The monopolar electrosurgical electrode of any one of claims 1-9, wherein the electrically conductive material is stainless steel.

11. The monopolar electrosurgical electrode of any one of claims 1-10, wherein the first electrode sensor is a thermocouple.

12. The monopolar electrosurgical electrode of any one of claims 1-11, wherein the first electrode sensor comprises a depth sensor that is configured to sense a depth that the electrosurgical blade is inserted into tissue.

13. A method of forming a monopolar electrosurgical electrode, comprising:
forming an electrosurgical substrate from an electrically conductive material, wherein the electrosurgical substrate comprises an electrosurgical blade, wherein the electrosurgical blade comprises:
a first lateral surface,
a second lateral surface opposite the first lateral surface,
a first major surface extending between the first lateral surface and the second lateral surface on a first side of the electrosurgical blade, and
a second major surface extending between the first lateral surface and the second lateral surface on a second side of the electrosurgical blade that is opposite the first side;
forming a first electrical insulation layer on the first major surface the electrosurgical blade;
forming a first electrode sensor on the first electrical insulation layer on the first major surface; and
forming a second electrical insulation layer on the first electrode sensor and the first electrical insulation layer such that the first electrode sensor is embedded between the first electrical insulation layer and the second electrical insulation layer.

14. The method of claim 13, wherein forming the electrosurgical substrate comprises cutting the electrosurgical substrate out of a wafer that comprises a plurality of other electrosurgical substrates.

15. The method of claim 14, wherein the wafer comprises a stainless steel wafer.

16. The method of any one of claims 13-15, wherein the wafer has a thickness of approximately 500 microns.

17. The method of any of claims 13-16, wherein the wafer has a diameter of approximately 200 millimeters (mm).

18. The method of any one of claims 13-17, wherein forming the first electrical insulation layer comprises at least one of performing at least one process selected from a group consisting of: (i) a plasma vapor deposition process, (ii) a chemical vapor deposition process, (iii) a printing process, (iv) a spin coating process, (v) a spray coating process, (vi) a sintering process, and (vii) a thermal oxidation process.

19. The method of any one of claims 13-18, wherein forming the first electrode sensor comprises forming the first electrode sensor using a lithography process.

20. The method of claim 19, wherein forming the first electrode sensor using the lithography process comprises:
applying a layer of adhesive to the first electrical insulation layer,
applying a lithographic photomask to the first insulation layer, wherein the lithographic photomask defines an exposed portion on the first electrical insulation layer;
after applying the lithographic photomask, forming one or more conductive traces in the exposed portion on the first electrical insulation layer; and
after forming the one or more conductive traces, removing the lithographic photomask from the first electrical insulation layer without removing the one or more conductive traces.

21. The method of forming an electrosurgical electrode of any one of claims 19-20, further comprising:
forming one or more alignment marks on the wafer; and
performing, based on the one or more alignment marks, the lithography process.

22. The method of claim 21, wherein forming the first electrode sensor comprises:
forming the first electrode sensor on a silicon carrier wafer;
after forming the first electrode sensor on the silicon carrier wafer, transferring the first electrode sensor to the electrosurgical substrate; and
after transferring the first electrode sensor to the electrosurgical substrate, cutting the electrosurgical substrate out of a wafer made from the electrically conductive material.

23. The method of any one of claims 13-22, further comprising:
forming a third electrical insulation layer on the second major surface the electrosurgical blade;
forming a second electrode sensor on the third electrical insulation layer on the second major surface; and
forming a fourth electrical insulation layer on the second electrode sensor and the third electrical insulation layer such that the second electrode sensor is embedded between the third electrical insulation layer and the fourth electrical insulation layer.

24. The method of claim 23, wherein forming the first electrode sensor comprises forming a temperature sensor, and
wherein forming the second electrode sensor comprises forming an electrochemical sensor.

25. The method of any one of claims 13-24, wherein forming the first electrode sensor comprises forming at least one sensor selected from a group of sensors consisting of: (i) a temperature sensor, (ii) an electrochemical sensor, (iii) a force sensor, (iv) a mass loading sensor, (v) a dielectric sensor, (vi) a conductivity sensor, (vii) a metal detector sensor, (viii) a tracking sensor, (ix) a light sensor, and (x) a smoke sensor.

26. A method of performing electrosurgery, comprising:
coupling an electrosurgical tool to an electrosurgical generator, wherein the electrosurgical tool comprises a monopolar electrosurgical electrode, wherein the monopolar electrosurgical electrode comprises:
an electrosurgical substrate comprising an electrically conductive material extending in an axial direction from a proximal end to a distal end, wherein the proximal end is configured to receive electrosurgical energy from an electrosurgical tool, wherein the electrosurgical substrate comprises an electrosurgical blade that is configured for at least one of cutting or coagulation of tissue by the electrosurgical energy received from the electrosurgical tool, wherein the electrosurgical blade comprises:
(i) a first lateral surface,
(ii) a second lateral surface opposite the first lateral surface,
(iii) a first major surface extending between the first lateral surface and the second lateral surface on a first side of the electrosurgical blade, and
(iv) a second major surface extending between the first lateral surface and the second lateral surface on a second side of the electrosurgical blade that is opposite the first side, and
a first electrode sensor embedded between a plurality of electrical insulation layers on the first major surface of the electrosurgical blade;
receiving, by the electrosurgical tool, the electrosurgical energy from the electrosurgical generator;
responsive to receiving the electrosurgical energy, performing, using the monopolar electrosurgical electrode, an electrosurgical operation; and
sensing, using the first electrode sensor, a condition related to the electrosurgical operation.

27. The method of claim 26, wherein the first electrode sensor is a temperature sensor, and
wherein sensing the condition comprises measuring a temperature of the monopolar electrosurgical electrode.

28. The method of claim 27, further comprising:
performing a comparison of the temperature measured by the temperature sensor with a threshold value;
determining, based on the comparison, that the temperature is greater than the threshold value; and
responsive to determining that the temperature is greater than the threshold value, halting a supply of the electrosurgical energy from the electrosurgical generator to the electrosurgical tool.

29. The method of claim 28, wherein the threshold value is less than or equal to a melting temperature of at least one of the plurality of electrical insulation layers.

30. The method of claim 28, further comprising setting the threshold value to a value that is less than a temperature at which tissue undergoes necrosis.

31. The method of any one of claims 26-30, further comprising:
determining, based on the condition sensed by the first electrode sensor, that the monopolar electrosurgical electrode is within a predetermined proximity of metal; and
responsive to determining that the monopolar electrosurgical electrode is within the predetermined proximity of metal, generating at least one alert selected from a group consisting of: an audible alert, a visual alert, and a haptic alert.

32. The method of any one of claims 26-31, further comprising detecting, based on the condition sensed by the first electrode sensor, a presence of a cancer marking dye,
wherein the first electrode sensor is an electrochemical sensor.

33. The method of any one of claims 26-32, wherein sensing the condition comprises measuring, using the first electrode sensor, an impedance of tissue, and
wherein the first electrode sensor is an electrochemical sensor.

34. The method of claim 33, wherein measuring the impedance of the tissue is performed while simultaneously cutting the tissue using the electrosurgical energy.

35. The method of any one of claims 26-34, wherein sensing the condition comprises sensing, using the first electrode sensor, a presence of dead tissue, and
wherein the first electrode sensor is an electrochemical sensor.

36. The method of any one of claims 26-35, wherein sensing the condition comprises detecting, using the first electrode sensor, a presence of alcohol, and
wherein the first electrode sensor is an electrochemical sensor.

37. The method of any one of claims 26-36, further comprising sensing a resistance of one or more resistive elements of the first electrode sensor and, based on the resistance, determining at least one item of information selected from a group including: (i) a type of the monopolar electrosurgical electrode, (ii) a size of the monopolar electrosurgical electrode, (iii) a shape of the monopolar electrosurgical electrode, (iv) a manufacturer of monopolar electrosurgical electrode, and (v) a number of times the monopolar electrosurgical electrode has used.

38. The method of any one of claims 26-37, wherein sensing the condition comprises measuring, using the first electrode sensor, a mass loading on the monopolar electrosurgical electrode due to tissue adhered to the monopolar electrosurgical electrode.

39. The method of any one of claims 26-38, wherein sensing the condition comprises measuring, using the first electrode sensor, a force applied to the monopolar electrosurgical electrode.

40. The method of any one of claims 26-39, wherein sensing the condition comprises measuring, using the first electrode sensor, an intensity of light incident on the monopolar electrosurgical electrode, and
wherein the first electrode sensor is a light sensor.

41. The method of claim 40, further comprising adjusting a power supplied to a light source based on the intensity of light measured by the light sensor.

42. The method of any one of claims 26-41, wherein sensing the condition comprises measuring, using the first electrode sensor, an amount of surgical smoke.

43. The method of claim 42, further comprising adjusting an amount of suction at a surgical site based on the amount of surgical smoke measured by the first electrode sensor.

44. The method of any one of claims 26-34, wherein sensing the condition comprises sensing, using the first electrode sensor, at least one of a location, an inertia, or an orientation of the monopolar electrosurgical electrode.

45. The method of any one of claims 26-44, wherein the monopolar electrosurgical electrode further comprises a second electrode sensor embedded between the plurality of electrical insulation layers on the second major surface of the electrosurgical blade, and
wherein the method further comprises sensing, using the second electrode sensor, a second condition related to the electrosurgical operation.

46. The method of claim 45, wherein the first electrode sensor comprises a temperature sensor and the second electrode sensor comprises an electrochemical sensor.
